(19) United States
(12) Patent Application Publication
(10) Pub. No.: US 2014/0212575 A1
(43) Pub. Date: Jul. 31, 2014

(54) Novel Self-Assembling Nanocomposite Structures and Methods of Preparing Same

(71) Applicant: The Regents of the University of Colorado, a body corporate, Denver, CO (US)

(72) Inventors: Wei Zhang, Superior, CO (US); Ryan McCaffrey, Boulder, CO (US); Wounjhang Park, Superior, CO (US); Kazunori Emoto, Broomfield, CO (US); Yinghua Jin, Superior, CO (US)

(21) Appl. No.: 14/035,308

(22) Filed: Sep. 24, 2013

Related U.S. Application Data
(60) Provisional application No. 61/847,207, filed on Jul. 17, 2013.
(60) Provisional application No. 61/704,907, filed on Sep. 24, 2012.

(51) Int. Cl. C07D 487/16; C07F 1/00

(52) U.S. Cl. CPC C07D 487/16; C07F 1/00
USPC 427/71; 540/472; 540/465; 427/197; 427/131; 427/199

(57) ABSTRACT

The present invention includes a novel self-assembling nanocomposite structure comprising a shape-persistent three-dimensional cage molecule. In one aspect, binding of nanoparticles to metal coordinating groups in the cage molecule allows for the self-assembly of the nanoparticles into a nanocomposite material. The present invention further includes methods of preparing such self-assembling nanocomposite structures.

### CROSS-REFERENCE TO RELATED APPLICATIONS

The present application claims priority under 35 U.S.C. §119(e) to U.S. Provisional Applications No. 61/704,907, filed Sep. 24, 2012, and No. 61/847,207, filed Jul. 17, 2013, all of which applications are hereby incorporated by reference in their entireties herein.

### STATEMENT REGARDING FEDERALLY SPONSORED RESEARCH OR DEVELOPMENT

This invention was made with government support under grant number D11AP00284 awarded by DOD/DARPA and grant number W911NF-12-1-0581 awarded by the U.S. Army Research Office. The government has certain rights in the invention.

### BACKGROUND OF THE INVENTION

Artificial structured materials, such as photonic crystals and metamaterials, hold high promise of providing a path to novel optical materials with rationally engineered optical properties. In the past decade, the field has experienced much growth, which was fueled by identification of novel properties such as negative refractive index and invisibility. One of the major roadblocks for development of such artificial structured materials is the lack of efficient fabrication technology, a grand challenge in the field of nanotechnology in general. The majority of optical metamaterials prepared so far have been fabricated by top-down techniques such as electron-beam lithography or focused ion beam milling. While these techniques may be used to sculpt nanostructures with high precision, they are slow, expensive and generally limited to two-dimensional structures. An efficient and scalable technique capable of producing large-area three-dimensional (3D) structures is critically needed.

Self-assembly of nanoparticles could offer an attractive solution to this problem. The simplest approach of self-assembling nanoparticles is to use hard sphere interaction. When the nanoparticles are highly monodispersed, the hard sphere interaction could lead to close-packing of nanoparticles into the face centered cubic structure (Park & Xia, 1999, Langmuir 15:266-273), which could also be achieved by controlled drying of solvent (Jiang et al., 1999, Chem. Mater. 11:2132-2140). While these methods provide simple and effective ways to obtain large-size, 3D-ordered structures, they generally produce large number of defects, and as a consequence long-range order has yet to be achieved by these techniques.

Furthermore, self-assembly of plasmonic nanoparticles presents an additional challenge. Generally, plasmonic nanoparticle volume fraction has to be on the order of 1-10% to generate interesting optical phenomena. However, such high concentration is difficult to achieve due to the strong van der Waals interaction between metal nanoparticles, which tends to lead to irreversible agglomeration (Lee et al., 2006, J. Mater. Res. 21:3215-3221). In a typical organic solvent, maximum achievable volume fraction of gold nanoparticles is less than 0.1% (Kubo et al., 2007, Nano Lett. 7:3418-3423). It is thus necessary to find a way to overcome the short-range van der Waals force. One way is to increase the electrostatic repulsion by coating the particle surface with electrically charged molecules (Lee & Park, 2008, Funct. Mater. Lett. 1:65-69). Alternatively, one can incorporate thin coatings that provide strong steric hindrance. Recently, successful self-assembly of gold and silver nanoparticles using template-directed self-assembly was demonstrated (Lee et al., 2009, Opt. Lett. 34:443-445; Tamma et al., 2010, Appl. Opt. 49:A11-A17). In this approach, a nanotemplate was first fabricated by laser interference lithography (a technique capable of generating highly ordered nanoscale patterns over centimeter-scale size). The nanopatterns on the template were chosen to produce desired electric or magnetic responses. Gold and silver nanoparticles were then self-assembled to fill the nanopatterns on the template, producing self-assembled metamaterial structures that exhibited electric and magnetic resonances as designed. However, the self-assembled nanoparticle clusters showed up to ˜18% variations in the cluster size, due primarily to the colloidal instability persistent in the system even when using inert coatings.

A recent work on gold nanoparticle dispersion in smectic A liquid crystal matrix showed that highly uniform and stable dispersion of gold nanoparticles was possible at extremely high volume fractions (up to 50%) (Pratibha et al., 2009, Opt. Express 17:19459-19469). In this system, the strong curvature in molecular alignment created by the presence of nanoparticles produced repulsive force between the nanoparticles strong enough to overcome the van der Waals attractive force, in a remarkably similar mechanism for the stable dislocation known to be present in smectic A liquid crystals (Pratibha et al., 2010, J. Appl. Phys. 107:063511).

The size-dependent optical and electronic properties of gold nanoparticles (Au NPs) have long been of interest in the context of nanoscience and nanotechnology (Zhao et al., 2013, Coordin. Chem. Rev. 257:638-665; Daniel and Astruc, 2004, Chem. Rev. 104:293-346). In particular, Au NPs <2 nm in diameter are known to exhibit properties that are quite unique compared to those larger than 5 nm (Aikens, 2011, J. Phys. Chem. Lett. 2:99-104), an observation that has motivated intense interest in the design of organic architectures for the template synthesis of 1-2 nm Au NPs that can be further used in catalysis (Helms and Frechet, 2006, Adv. Synth. Catal. 348:1125-1148; Myers et al., 2011, Chem. Sci. 2:1632-1646; Astruc et al., 2012, Acc. Chem. Res. 45:630-640), sensor devices (Lim and Zhong, 2009, Acc. Chem. Res. 42:798-808), and nanoelectronics (Homberger and Simon, 2010, Phil. Trans. R. Soc. A 368:1405-1453). For instance, the realization of single-electron devices will require the use of nanoparticles with diameters below 2 nm (Schmid and Simon, 2005, Chem. Commun. 697-710).

Since the advent of the Brust-Schiffrin method (Brust et al., 1994, J. Chem. Soc. Chem. Commun. 801-802), there has been a wealth of literature exploring the structure-function relationships of ligand stabilized nanoparticles that can be rationally designed for fundamental studies and practical applications (Scott et al., 2005, J. Phys. Chem. B 109:692-704; Thompson et al., 2012, Acs Nano 6:3007-3017; Westerlund and Bjornholm, 2009, Curr. Opin. Colliod Interface Sci. 14:126-134; Whitesides and Grzybowski, 2002, Science 295:2418-2421; Whitesides et al., 2005, pp. 217-239, in Nanoscale Assembly Springer, New York). Such ligands must have (i) a well-defined architecture, (ii) the ability to control monodispersity by avoiding random nucleation and growth and (iii) the capability to achieve desired structures and functionalities. Control over encapsulation and monodispersity is usually a function of ‘templated-synthesis’ (Kim et al., 2004, Chem. Mater. 16:167-172), and to date most substrate encapsulation efforts have advanced through the use of dendritic architectures as a way of controlling nanoparticle size and shape variations, and distribution (Myers et al., 2011, Chem. Sci. 2:1632-1646; Esumi et al., 2000, Langmuir 16:2604-2608; Grohn et al., 2000, Macromolecules 33:6042-6050; Crooks et al., 2001, Acc. Chem. Res. 34:181-190; Vassilieff et al., 2008, J. Mater. Chem. 18:4031-4033; Boisselier et al., 2010, J. Am. Chem. Soc. 132:2729-2742; Hermes et al., 2011, Chem. Eur. J. 17:13473-13481; D'Aleo et al., 2004, Adv. Func. Mater. 14:1167-1177). Despite the successes of dendritic-based ligands, there still remain very few examples of passivating ligands allowing for a size-controlled synthesis of gold nanoparticles. Zhang and coworkers have described organic porous materials comprising shape-persistent 3D molecular cage building blocks in U.S. Patent Application Publication No. US 2013/0047849.

There is a need in the art for novel methodologies for achieving self-assembly of nanoparticles, such as metal nanoparticles, magnetic nanoparticles, fluorescent nanoparticles, semiconductor nanoparticles, quantum dot nanoparticles, dielectric nanoparticles, or any combinations thereof. Such methodologies should afford precise and pre-determined control of interparticle spacing down to the molecular level. Such nanometric control over the self-assembled structures should allow for the synthesis of nanocomposite structures with novel macroscopic properties. The present invention fulfills this need.

### BRIEF SUMMARY OF THE INVENTION

The invention includes a composition comprising a shape-persistent three-dimensional cage molecule, wherein the cage molecule comprises two or more metal coordinating groups. In one embodiment, binding of nanoparticles to the two or more metal coordinating groups allows for the self-assembly of the nanoparticles into a nanocomposite material.

In one embodiment, the nanoparticles comprise a metal nanoparticle, magnetic nanoparticle, fluorescent nanoparticle, semiconductor nanoparticle, quantum dot nanoparticle, dielectric nanoparticle, or any combinations thereof. In another embodiment, the cage molecule is selected so that the nanoparticles in the nanocomposite material are held at a pre-determined spacing. In yet another embodiment, the nanocomposite material is deposited on at least a portion of the surface of a solid substrate.

In one embodiment, the cage molecule has a polyhedron shape selected from the group consisting of trigonal prismatic, tetrahedron, octahedron, cubic, dodecahedron, pentagonal prismatic, hexagonal prismatic, and any combinations thereof. In another embodiment, the cage molecule is selected from the group consisting of formulas (I)-(III), any combinations thereof, and any salts thereof:

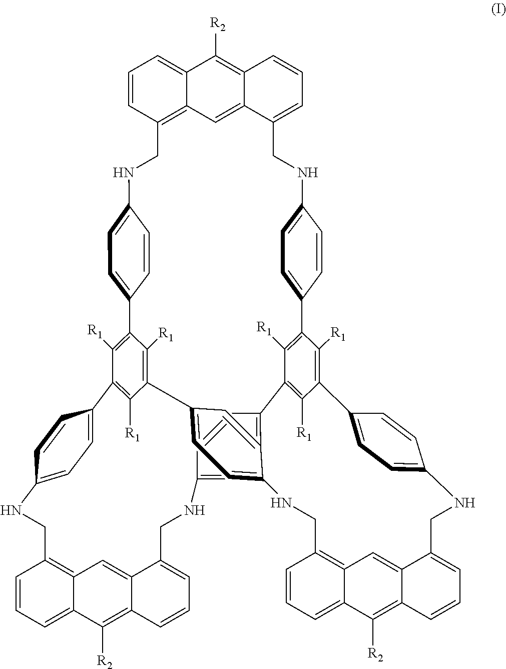

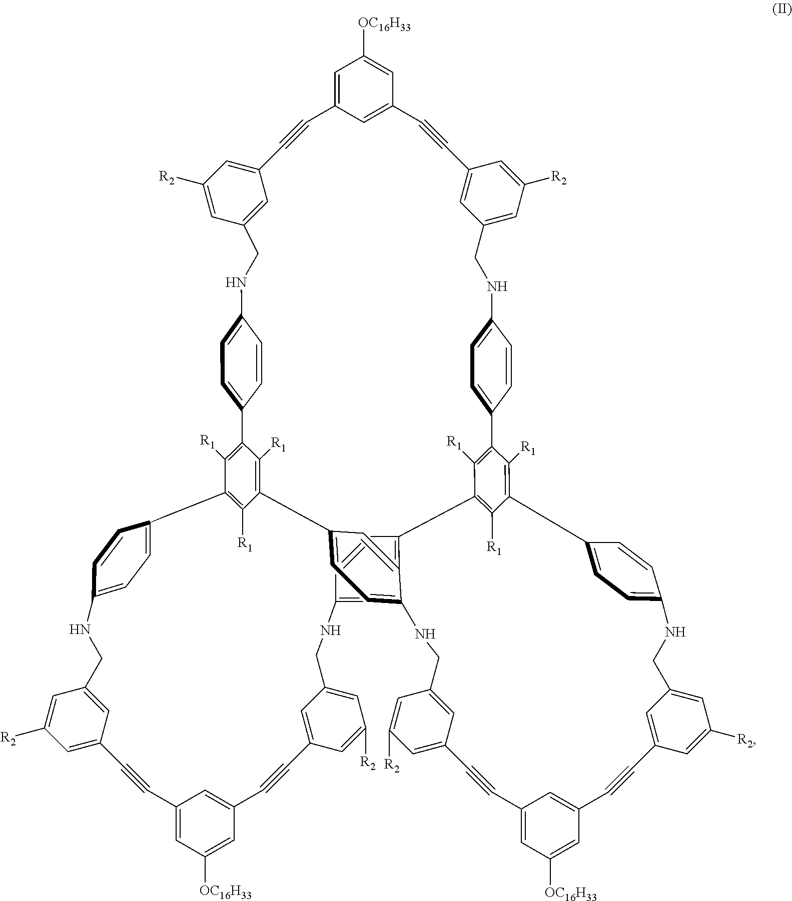

wherein in formulas (I)-(II) each occurrence of R₁ is independently C₂-C₁₀ alkyl; each occurrence of R₂ comprises a metal coordinating group and is independently selected from the group consisting of aryl, heteroaryl, arylalkyl, arylalkenyl, arylalkynyl, heteroarylalkyl, heteroarylalkenyl, heteroarylalkynyl, heterocyclyl, heterocyclylalkyl, heterocyclylalkenyl and heterocyclylalkynyl;
and,

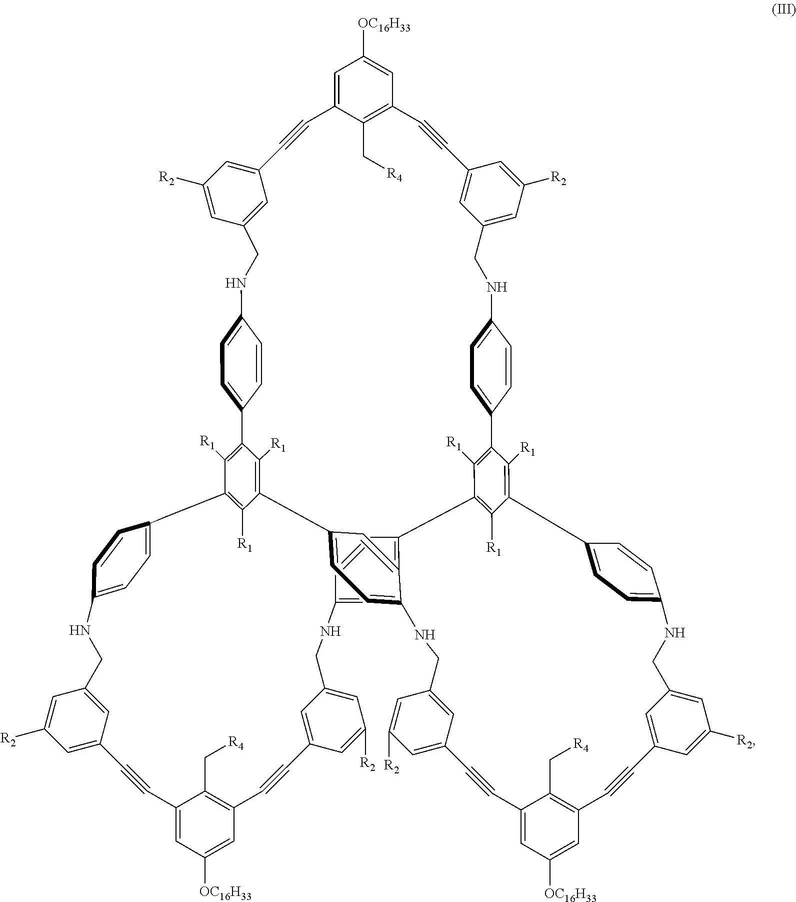

wherein in formula (III) each occurrence of R₁ is independently C₂-C₁₀ alkyl; each occurrence of R₂ is independently selected from the group consisting of hydrogen, aryl, heteroaryl, arylalkyl, arylalkenyl, arylalkynyl, heteroarylalkyl, heteroarylalkenyl, heteroarylalkynyl, heterocyclyl, heterocyclylalkyl, heterocyclylalkenyl and heterocyclylalkynyl; and each occurrence of R₄ is a metal coordinating group.

In one embodiment, in formulas (I)-(III) each occurrence of R₁ is independently C₄-C₈ alkyl. In another embodiment, in formulas (I)-(III) each occurrence of R₁ is C₆H₁₃.

In one embodiment, in formulas (I)-(II) the metal coordinating group in each occurrence of R₂ is independently selected from the group consisting of pyridyl, bipyridyl, terpyridyl, anilino, amino, carboxylate, phosphate, sulfate, amido, sulfonamido, hydroxy, sulfhydryl, and cyano. In another embodiment, in formulas (I)-(II) the metal coordinating group in each occurrence of R₂ is pyridyl. In yet another embodiment, in formulas (I)-(II) each occurrence of R₂ is independently pyridin-4-yl or 2-(pyridin-4-yl)vinyl.

In one embodiment, in formula (III) each occurrence of R₂ is hydrogen. In another embodiment, in formula (III) each occurrence of R₄ is independently selected from the group consisting of pyridyl, bipyridyl, terpyridyl, anilino, amino, carboxylate, phosphate, sulfate, amido, sulfonamido, hydroxy, sulfhydryl, and cyano. In yet another embodiment, in formula (III) each occurrence of R₄ is SR₃, wherein each occurrence of R₃ is independently H or C₁-C₁₀ alkyl. In yet another embodiment, in formula (III) each occurrence of R₃ is independently C₁-C₅ alkyl. In yet another embodiment, in formula (III) each occurrence of R₃ is C₂H₅.

In one embodiment, the cage molecule comprises COP-3P, COP-6VP, COP-11, any salt thereof, or any combinations thereof.

In one embodiment, the composition further comprises nanoparticles, wherein the nanoparticles are bound to the cage molecule through the two or more metal coordinating groups. In another embodiment, the nanoparticles comprise a metal nanoparticle, magnetic nanoparticle, fluorescent nanoparticle, semiconductor nanoparticle, quantum dot nanoparticle, dielectric nanoparticle, or any combinations thereof. In yet another embodiment, the metal nanoparticles comprise gold, silver, aluminum, copper, nickel, iron, chromium, titanium, platinum, cobalt, palladium, or any combinations thereof.

The invention also includes a method of preparing a self-assembling nanocomposite structure on at least a fraction of the surface of a solid substrate. In one embodiment, the method comprises the step of (a) providing a solid substrate. In another embodiment, the method comprises the step of (b) depositing a layer of nanoparticles on at least a fraction of the substrate surface. In yet another embodiment, the method comprises the step of (c) applying sequentially to the at least a fraction of the substrate surface a solution of a shape-persistent three-dimensional cage molecule and a solution of nanoparticles. In yet another embodiment, the cage molecule comprises two or more metal coordinating groups, whereby binding of the nanoparticles to the two or more metal coordinating groups allows for the self-assembly of the nanoparticles into a nanocomposite material. In yet another embodiment, the method comprises the step of (d) optionally removing excess solution from the at least a fraction of the substrate surface. In yet another embodiment, the method comprises the step of (e) repeating steps (c) and (d) until the desired thickness of the self-assembling nanocomposite structure on the substrate surface is obtained.

In one embodiment, the nanoparticles comprise a metal nanoparticle, magnetic nanoparticle, fluorescent nanoparticle, semiconductor nanoparticle, quantum dot nanoparticle, dielectric nanoparticle, or any combinations thereof.

In one embodiment, step (b) comprises contacting the at least a fraction of the substrate surface with nanoparticles of opposite charge. In another embodiment, the at least a fraction of the substrate surface is rendered positively charged by amine functionalization. In yet another embodiment, the applying of step (c) comprises drop casting.

In one embodiment, the cage molecule has a polyhedron shape selected from the group consisting of trigonal prismatic, tetrahedron, octahedron, cubic, dodecahedron, pentagonal prismatic, hexagonal prismatic, and any combinations thereof. In another embodiment, the cage molecule is selected from the group consisting of formulas (I)-(III), any combinations thereof, and any salts thereof:

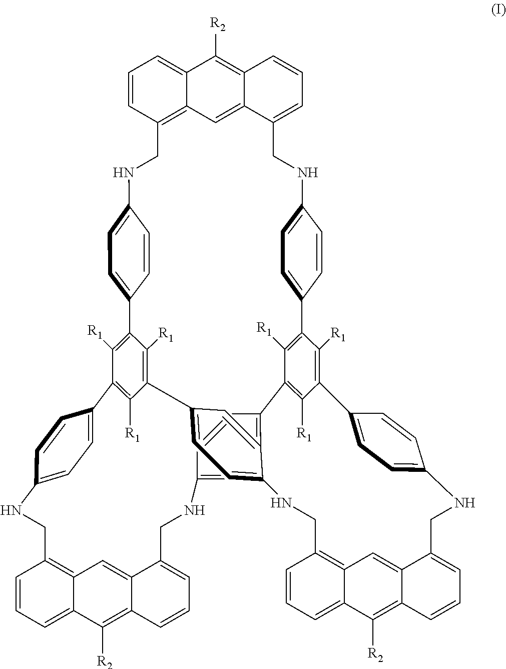

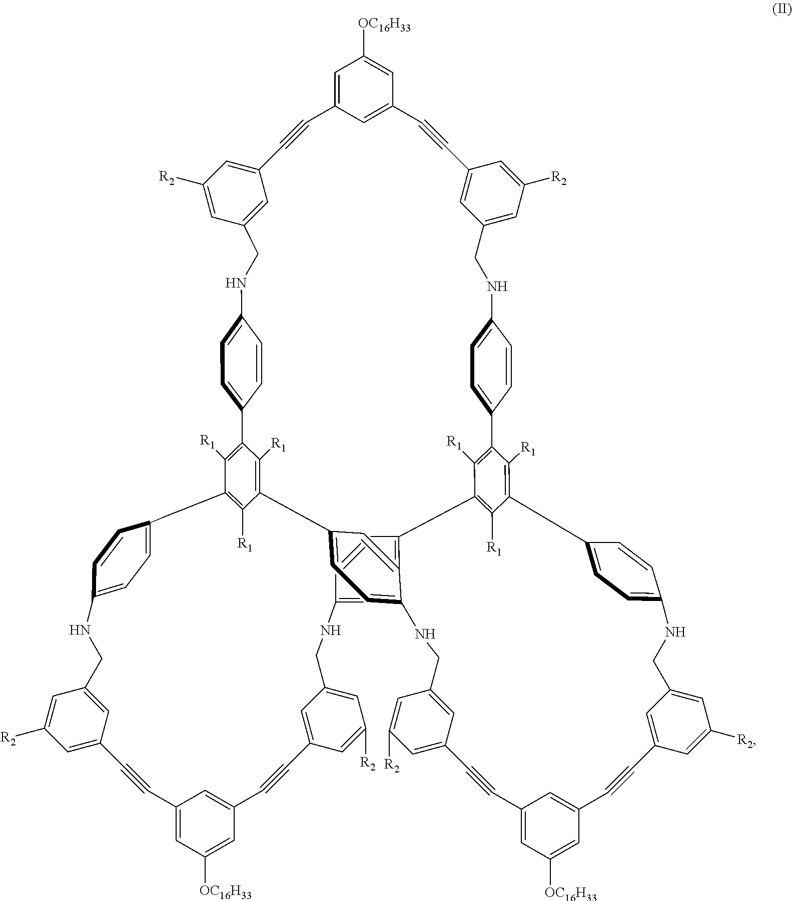

wherein in formulas (I)-(II) each occurrence of R₁ is independently C₂-C₁₀ alkyl; each occurrence of R₂ comprises a metal coordinating group and is independently selected from the group consisting of aryl, heteroaryl, arylalkyl, arylalkenyl, arylalkynyl, heteroarylalkyl, heteroarylalkenyl, heteroarylalkynyl, heterocyclyl, heterocyclylalkyl, heterocyclylalkenyl and heterocyclylalkynyl; and,

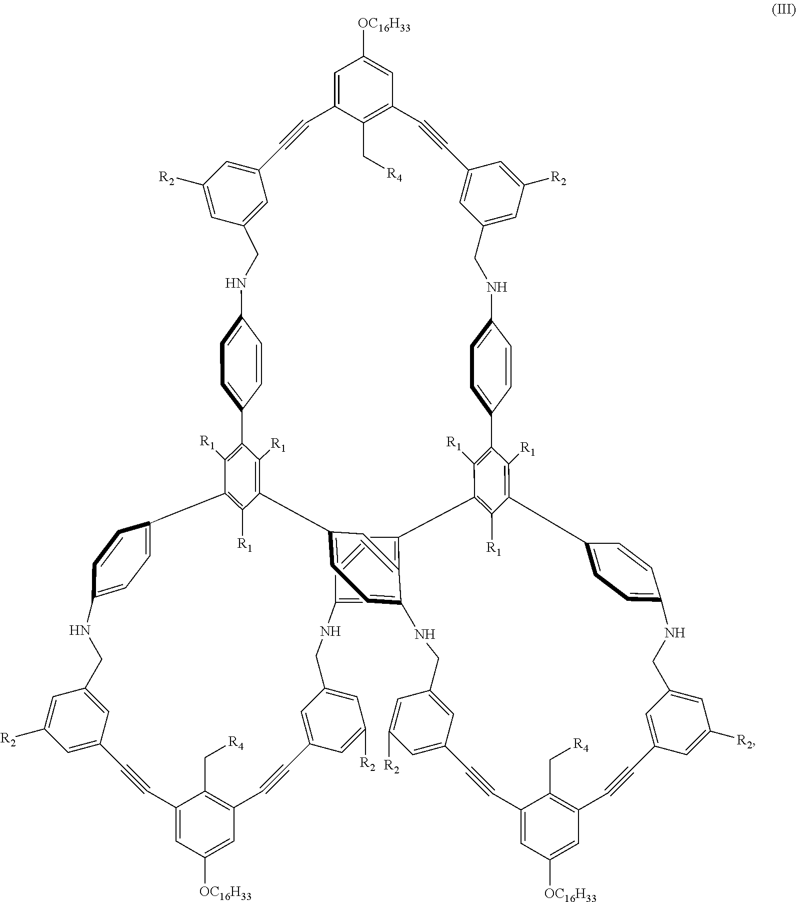

wherein in formula (III) each occurrence of R₁ is independently C₂-C₁₀ alkyl; each occurrence of R₂ is independently selected from the group consisting of hydrogen, aryl, heteroaryl, arylalkyl, arylalkenyl, arylalkynyl, heteroarylalkyl, heteroarylalkenyl, heteroarylalkynyl, heterocyclyl, heterocyclylalkyl, heterocyclylalkenyl and heterocyclylalkynyl; and each occurrence of R₄ is a metal coordinating group.

In one embodiment, in formulas (I)-(III) each occurrence of R₁ is independently C₄-C₈ alkyl. In another embodiment, in formulas (I)-(III) each occurrence of R₁ is C₆H₁₃.

In one embodiment, in formulas (I)-(II) the metal coordinating group in each occurrence of R₂ is independently selected from the group consisting of pyridyl, bipyridyl, terpyridyl, anilino, amino, carboxylate, phosphate, sulfate, amido, sulfonamido, hydroxy, sulfhydryl, and cyano. In another embodiment, in formulas (I)-(II) the metal coordinating group in each occurrence of R₂ is pyridyl. In yet another embodiment, in formulas (I)-(II) each occurrence of R₂ is independently pyridin-4-yl or 2-(pyridin-4-yl)vinyl.

In one embodiment, in formula (III) each occurrence of R₂ is hydrogen. In another embodiment, in formula (III) each occurrence of R₄ is independently selected from the group consisting of pyridyl, bipyridyl, terpyridyl, anilino, amino, carboxylate, phosphate, sulfate, amido, sulfonamido, hydroxy, sulfhydryl, and cyano. In yet another embodiment, in formula (III) each occurrence of R₄ is SR₃, wherein each occurrence of R₃ is independently H or C₁-C₁₀ alkyl. In yet another embodiment, in formula (III) each occurrence of R₃ is independently C₁-C₅ alkyl. In yet another embodiment, in formula (III) each occurrence of R₃ is C₂H₅.

In one embodiment, the cage molecule comprises COP-3P, COP-6VP, COP-11, any salt thereof, or any combinations thereof.

In one embodiment, the composition further comprises nanoparticles, wherein the nanoparticles are bound to the cage molecule through the two or more metal coordinating groups. In another embodiment, the nanoparticles comprise a metal nanoparticle, magnetic nanoparticle, fluorescent nanoparticle, semiconductor nanoparticle, quantum dot nanoparticle, dielectric nanoparticle, or any combinations thereof. In yet another embodiment, the metal nanoparticles comprise gold, silver, aluminum, copper, nickel, iron, chromium, titanium, platinum, cobalt, palladium, or any combinations thereof.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 illustrates the synthesis of COP-3P and COP-6VP.

FIG. 2 illustrates molecules used for self-assembly of gold nanoparticles.

FIG. 3, comprising FIGS. 3A-3C, illustrates the characterization of gold nanoparticle layers. Topography scans obtained by AFM for (FIG. 3A) monolayer of gold nanoparticles and (FIG. 3B) 4 layers of gold nanoparticles self-assembled by COP-3P molecules. (FIG. 3C) Thickness measured by AFM as a function of the number of gold nanoparticle layers for self-assembly mediated by various molecules.

FIG. 4, comprising FIGS. 4A-4C, illustrates optical extinction spectra for self-assembled gold nanoparticles using linker molecules (FIG. 4A) TP, (FIG. 4B) COP-3P, and (FIG. 4C) COP-6VP.

FIG. 5, comprising FIGS. 5A-5D, illustrates transmission electron micrographs of gold nanoparticle clusters with various linker molecules: (FIG. 5A) bare nanoparticles, (FIG. 5B) TP, (FIG. 5C) COP-3P, and (FIG. 5D) COP-6VP. The scale bars indicate 20 nm.

FIG. 6, comprising FIGS. 6A-6C, illustrates experimental optical extinction spectra and the effective medium theory fitting for 3-layer and 4-layer self-assembled gold nanoparticle films using (FIG. 6A) TP, (FIG. 6B) COP-3P, and (FIG. 6C) COP-6VP.

FIG. 7, comprising FIGS. 7A-7C, illustrates real (∈ᵣ) and imaginary (∈ᵢ) parts of permittivity extracted from the effective medium theory fitting presented in FIG. 6. 3-layer samples were plotted with solid lines and 4-layer samples were plotted with symbols.

FIG. 8, comprising FIGS. 8A-8B, illustrates real (n) and imaginary (κ) parts of refractive index calculated from the effective medium theory (solid lines) and the multiple scattering theory (symbols) for the 4-layer samples prepared with (FIG. 8A) COP-3P and (FIG. 8B) COP-6VP.

FIG. 9, comprising FIGS. 9A-9C, is a set of graphs illustrating titration studies: TP (50 μM) (FIG. 9A), COP-3P (50 μM) (FIG. 9B), and COP-6VP (50 μM) (FIG. 9C) in THF were added (0-25 μL in 5 μL increments) to aqueous solutions of AuNPs (0.5 mL). After mixing the components, they were diluted to 5 mL and the absorption spectra were recorded.

FIG. 10 is a graph illustrating refractive indexes of thin films of cage molecules measured by ellipsometry.

FIG. 11, comprising FIGS. 11A-11D, illustrates the structures of COP-11 (FIG. 11A), COP-12 (FIG. 11B), and a fully stretched model of COP-11: the side view (FIG. 11C), and the top view (FIG. 11D). A methyl group and hydrogen were used in the calculation instead of hexyl and OC₁₆H₃₃ groups respectively for simplification.

FIG. 12 illustrates an exemplary synthesis of molecular cage COP-11. FIG. 13, comprised of FIGS. 13A-13D, illustrates TEM micrographs (scale bar 20 nm) of AuNP@COP-11 complex (FIG. 13A) and AuNP@COP-12 complex (FIG. 13B). FIG. 13C is a graph illustrating the UV-Vis absorption spectra of gold complexes in CH₂Cl₂. FIG. 13D is a graph illustrating the size distribution of AuNP@COP-11 complex.

FIG. 14 is a graph illustrating the energy of COP-11 as a function of encapsulated nanoparticle radius.

FIG. 15 illustrates the calculated energy-minimized structure of AuNP@COP-11. AuNP radius is 8.65 Å.

### DETAILED DESCRIPTION OF THE INVENTION

The invention relates to the unexpected discovery that shape-persistent three-dimensional cage molecules may be used for the self-assembly of functional nanoparticles, such as but not limited to gold nanoparticles. In one aspect, the modular construction of cage molecules allows for precise control of interparticle spacing down to the molecular level. In another aspect, the ability to change the number and flexibility of binding sites provides a means to tune the self-assembly process.

As described herein, distinct types of cage molecules equipped with varying numbers of binding (i.e., coordinating) groups with varying flexibility were designed and synthesized. Optical and structural analysis showed that the interparticle spacing within the self-assembled structures was precisely controlled by the choice of the cage molecules. The new self-assembly approach of the invention, based on molecular cage linkers, thus provides nanometric control over the structure.

In a non-limiting aspect, the two or more metal coordinating groups may be located on the exterior of the cage molecule. In one embodiment, the cage molecule serves as a linker to mediate the nanoparticle assembly through cage-nanoparticle binding. This embodiment provides a “cage-between-nanoparticles” system.

In another non-limiting aspect, the two or more metal coordinating groups may be located in the interior of the cage molecule. In one embodiment, the cage molecule serves as a template, whereby the nanoparticle grows inside the cage molecule. Further self-assembly or covalent linking of cage molecules leads to the assembly of the nanoparticles that are bound inside the cage molecules. This embodiment provides a “nanoparticle-in-cage” system.

### DEFINITIONS

As used herein, each of the following terms has the meaning associated with it in this section.

As used herein, unless defined otherwise, all technical and scientific terms generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “comprising” includes “including,” “containing” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising,” particularly in a description of components of a composition or in a description of elements of a device, is understood to encompass those compositions and methods consisting essentially of and consisting of the recited components or elements.

As used herein, a “metal coordinating group” refers to a chemical group capable of forming a coordinating bond to a metal ion. Non-limiting examples of metal coordinating groups are thioether, pyridyl, bipyridyl, terpyridyl, anilino, amino, carboxylate, phosphate, sulfate, amido, sulfonamido, hydroxy, sulfhydryl, and cyano.

As used herein, the term “chelator” refers to a molecule comprising two or more metal coordinating groups, whereby the two or more metal coordinating groups may bind to a single metal ion to form a “chelate” or “chelating complex.”

As used herein, the term “scaffold” refers to a two-dimensional or a three-dimensional supporting framework. A scaffold can form a two- or three-dimensional structure of controlled mesh size. A monolayer is a non-limiting exemplary two-dimensional structure.

As used herein, the term “TP” refers to 1,3,5-tris[2-(pyridin-4-yl)vinyl]-benzene or a salt thereof.

As used herein, the term “COP-1” refers to the compound of formula (I) wherein R₁ is C₆H₁₃ and R₂ is Br, or a salt thereof.

As used herein, the term “COP-3P” refers to the compound of formula (I) wherein R₁ is C₆H₁₃ and R₂ is pyridin-4-yl, or a salt thereof:

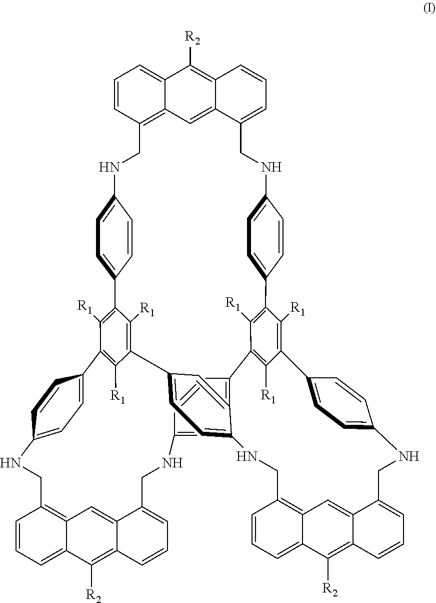

As used herein, the term “COP-2” refers to the compound of formula (II) wherein R₁ is C₆H₁₃ and R₂ is Br, or a salt thereof.

As used herein, the term “COP-6VP” refers to the compound of formula (II) wherein R₁ is C₆H₁₃ and R₂ is 2-(pyridin-4-yl)vinyl, or a salt thereof:

As used herein, the term “COP-11” refers to the compound of formula (III) wherein R₁ is C₆H₁₃, R₂ is hydrogen, and R₄ is SR₃, wherein R₃ is C₂H₅, or a salt thereof:

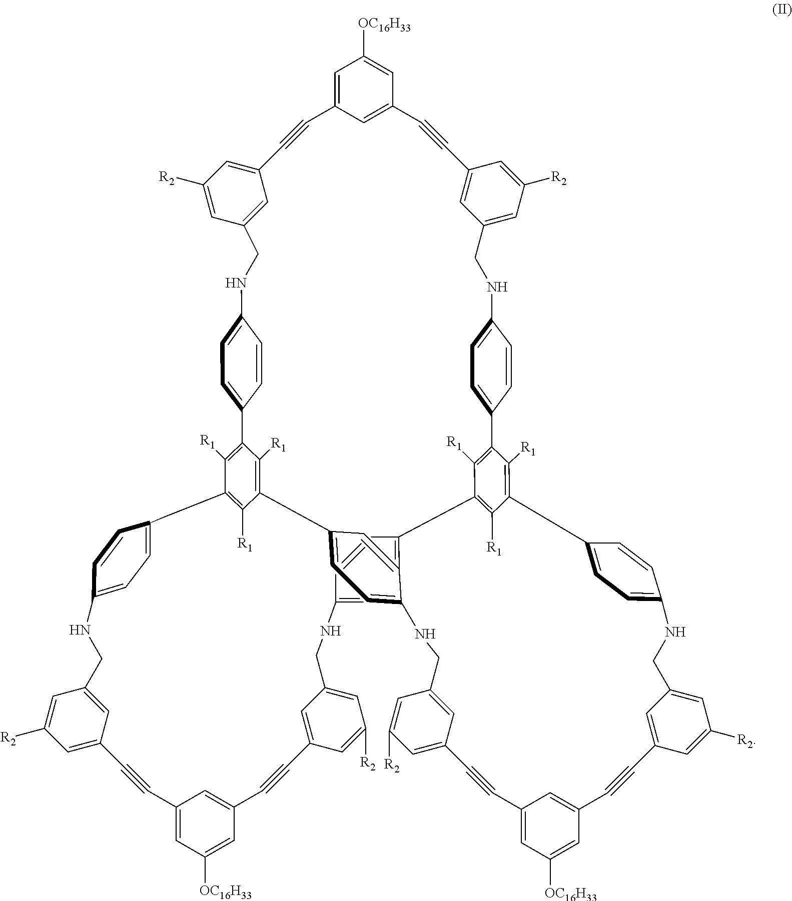

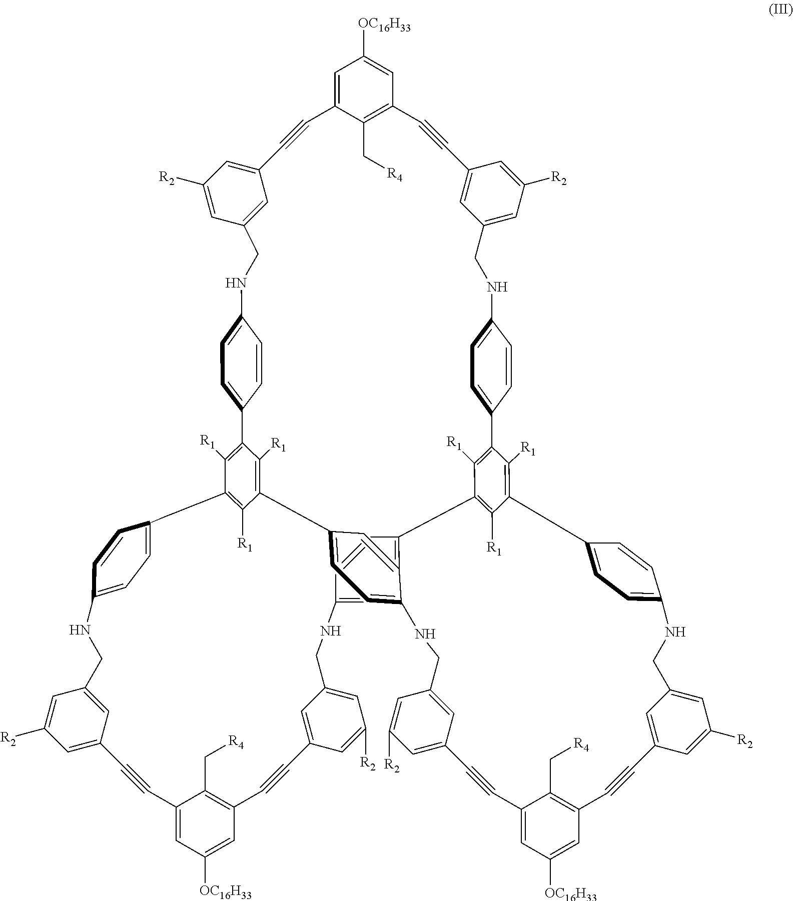

As used herein, the term “salt” refers to a salt of a compound contemplated within the invention, including inorganic acids, organic acids, inorganic bases, organic bases, solvates, hydrates, or clathrates thereof. As used herein, the term “salt” embraces addition salts of free acids or free bases that are compounds useful within the invention. Suitable acid addition salts may be prepared from an inorganic acid or an organic acid. Examples of inorganic acids include hydrochloric, hydrobromic, hydriodic, nitric, carbonic, sulfuric, phosphoric acids, perchloric and tetrafluoroboronic acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Suitable base addition salts of compounds useful within the invention include, for example, metallic salts including alkali metal, alkaline earth metal and transition metal salts such as, for example, lithium, calcium, magnesium, potassium, ammonium, sodium and zinc salts. Acceptable base addition salts also include organic salts made from basic amines such as, for example, N,N′-dibenzylethylenediamine, chloroprocaine, choline, diethanolamine, ethylenediamine, meglumine (N-methyl-glucamine) and procaine. All of these salts may be prepared by conventional means from the corresponding free base compound by reacting, for example, the appropriate acid or base with the corresponding free base.

As used herein, the term “instructional material” includes a publication, a recording, a diagram, or any other medium of expression that may be used to communicate the usefulness of the compositions and methods of the invention. In one embodiment, the instructional material may be part of a kit useful for generating a composition of the invention. The instructional material of the kit may, for example, be affixed to a container that contains the compositions of the invention or be shipped together with a container that contains the compositions. Alternatively, the instructional material may be shipped separately from the container with the intention that the recipient uses the instructional material and the compositions cooperatively. For example, the instructional material is for use of a kit; instructions for use of the compositions; or instructions for use of a formulation of the compositions.

As used herein, the term “alkyl,” by itself or as part of another substituent means, unless otherwise stated, a straight or branched chain hydrocarbon having the number of carbon atoms designated (i.e., C₁-C₆ means one to six carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆)alkyl, particularly ethyl, methyl, isopropyl, isobutyl, n-pentyl, n-hexyl and cyclopropylmethyl.

As used herein, the term “substituted alkyl” means alkyl as defined above, substituted by one, two or three substituents selected from the group consisting of halogen, —OH, alkoxy, —NH₂, —N(CH₃)₂, —C(═O)OH, —CF₃, —C≡N (or —CN), —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, —CF₃, —N(CH₃)₂, —C(═O)NH₂ and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” refers to a mono cyclic or polycyclic non-aromatic radical, wherein each of the atoms forming the ring (i.e., skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

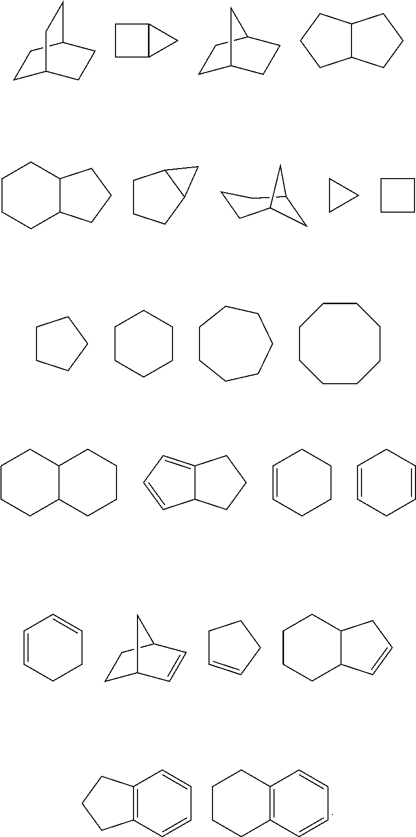

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon carbon double bond or one carbon carbon triple bond.

As used herein, the term “heterocycloalkyl” or “heterocyclyl” refers to a heteroalicyclic group containing one to four ring heteroatoms each selected from O, Sand N. In one embodiment, each heterocycloalkyl group has from 4 to 10 atoms in its ring system, with the proviso that the ring of said group does not contain two adjacent O or S atoms. In another embodiment, the heterocycloalkyl group is fused with an aromatic ring. In one embodiment, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

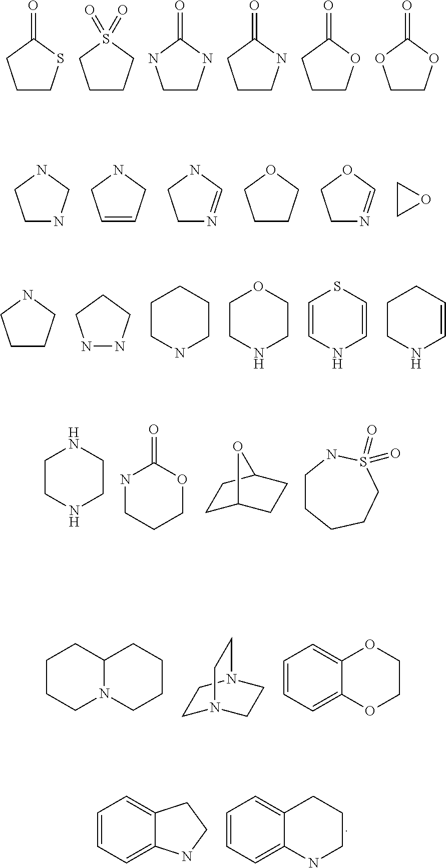

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e., having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl. Preferred examples are phenyl and naphthyl, most preferred is phenyl.

As used herein, the term “aryl-(C₁-C₃)alkyl” means a functional group wherein a one- to three-carbon alkylene chain is attached to an aryl group, e.g., —CH₂CH₂-phenyl. Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “heteroaryl” or “heteroaromatic” refers to a heterocycle having aromatic character. A polycyclic heteroaryl may include one or more rings that are partially saturated. Examples include the following moieties:

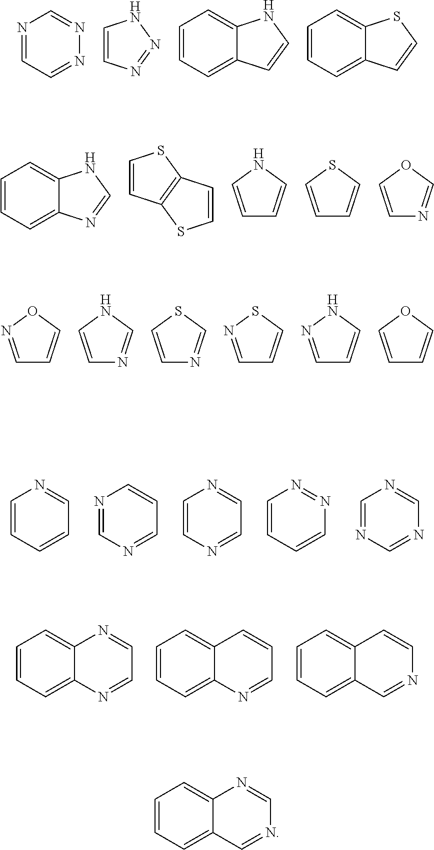

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, and —C(═O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

Throughout this disclosure, various aspects of the invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as, but not limited to, from 1 to 3, from 1 to 4, from 1 to 5, from 2 to 4, from 2 to 6, and from 3 to 6, as well as individual numbers within that range, for example, 1, 2, 2.7, 3, 4, 5, 5.3, and 6. This applies regardless of the breadth of the range.

### Compositions

The invention includes a composition comprising a shape-persistent three-dimensional cage molecule, wherein the cage molecule comprises two or more metal coordinating groups, whereby binding of nanoparticles to the two or more metal coordinating groups allows for the self-assembly of the nanoparticles into a nanocomposite material.

In a non-limiting aspect, the two or more metal coordinating groups may be located on the exterior of the cage molecule. In one embodiment, the cage molecule serves as a linker to mediate the nanoparticle assembly through cage-nanoparticle binding. This embodiment provides a “cage-between-nanoparticles” system.

In another non-limiting aspect, the two or more metal coordinating groups may be located in the interior of the cage molecule. In one embodiment, the cage molecule serves as a template, whereby the nanoparticle grows inside the cage molecule. Further self-assembly or covalent linking of cage molecules leads to the assembly of the nanoparticles that are bound inside the cage molecules. This embodiment provides a “nanoparticle-in-cage” system.

In one embodiment, the nanoparticles comprise a metal nanoparticle, magnetic nanoparticle, fluorescent nanoparticle, semiconductor nanoparticle, quantum dot nanoparticle, dielectric nanoparticle, or any combinations thereof. In another embodiment, the cage molecule is selected so that the nanoparticles in the nanocomposite material are held at a pre-determined spacing. In yet another embodiment, the nanocomposite material is deposited on at least a portion of the surface of a solid substrate. In yet another embodiment, the cage molecule has a polyhedron shape selected from the group consisting of trigonal prismatic, tetrahedron, octahedron, cubic, dodecahedron, pentagonal prismatic, hexagonal prismatic, and any combinations thereof.

In one embodiment, the cage molecule is selected from the group consisting of formula (I), formula (II), any combinations thereof, and any salts thereof:

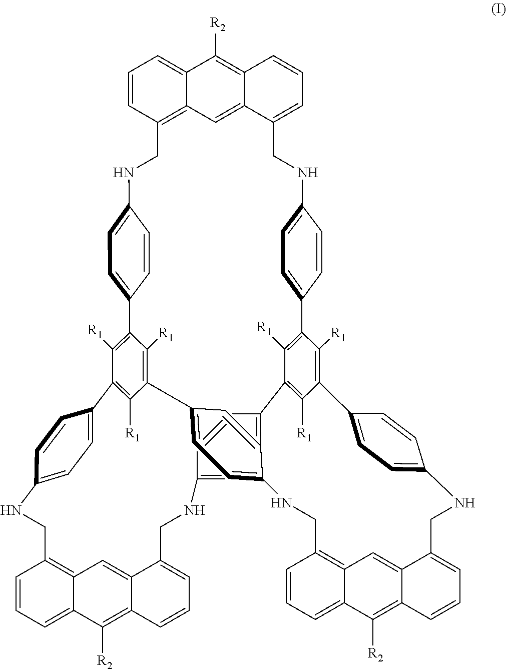

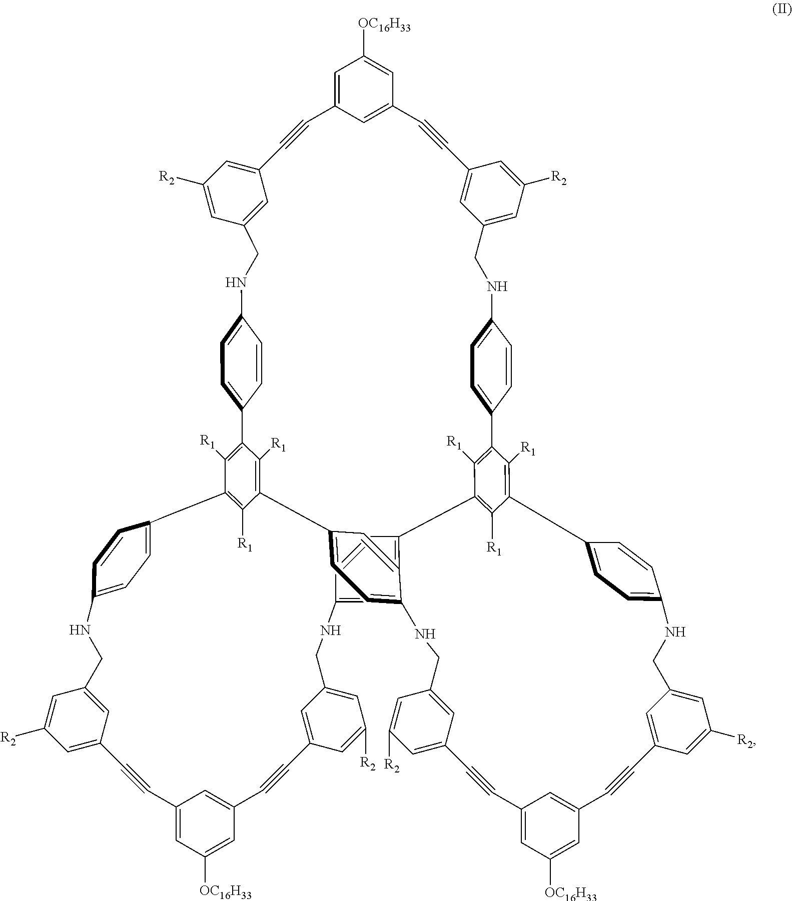

wherein each occurrence of R₁ is independently C₂-C₁₀ alkyl, and each occurrence of R₂ is independently selected from the group consisting of aryl, heteroaryl, arylalkyl, arylalkenyl, arylalkynyl, heteroarylalkyl, heteroarylalkenyl, heteroarylalkynyl, heterocyclyl, heterocyclylalkyl, heterocyclylalkenyl and heterocyclylalkynyl, further wherein R₂ comprises a metal coordinating group. In yet another embodiment, each occurrence of R₁ is independently C₄-C₈ alkyl. In yet another embodiment, each occurrence of R₁ is C₆H₁₃. In yet another embodiment, the metal coordinating group in each occurrence of R₂ is independently selected from the group consisting of pyridyl, bipyridyl, terpyridyl, anilino, amino, carboxylate, phosphate, sulfate, amido, sulfonamido, hydroxy, sulfhydryl, and cyano. In yet another embodiment, the metal coordinating group in each occurrence of R₂ is pyridyl. In yet another embodiment, each occurrence of R₂ is independently pyridin-4-yl or 2-(pyridin-4-yl)vinyl. In yet another embodiment, the cage molecule comprises COP-3P, COP-6VP, any salt thereof, or any combinations thereof. In yet another embodiment, the composition further comprises nanoparticles, wherein the nanoparticles are bound to the cage molecule through the two or more metal coordinating groups. In yet another embodiment, the nanoparticles comprise a metal nanoparticle, magnetic nanoparticle, fluorescent nanoparticle, semiconductor nanoparticle, quantum dot nanoparticle, dielectric nanoparticle, or any combinations thereof. In yet another embodiment, the metal nanoparticles comprise gold, silver, aluminum, copper, nickel, iron, chromium, titanium, platinum, cobalt, palladium, or any combinations thereof.

In one embodiment, the cage molecule is selected from the group consisting of formula (III), and any salts thereof:

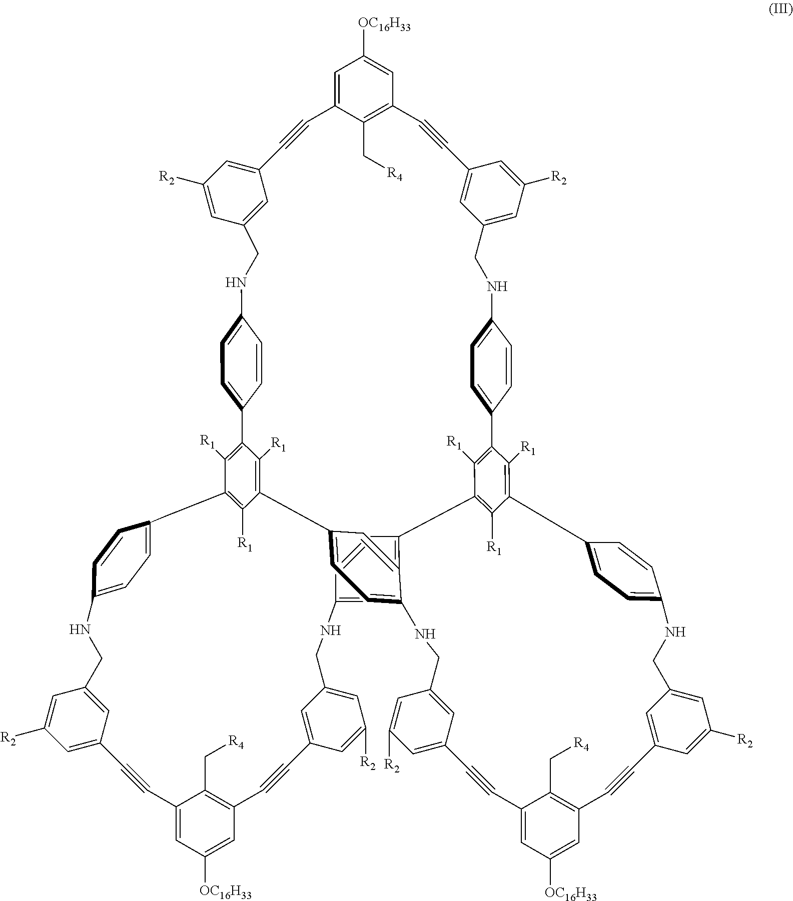

wherein for formula (III), each occurrence of R₁ is independently C₂-C₁₀ alkyl, each occurrence of R₂ is independently selected from the group consisting of hydrogen, aryl, heteroaryl, arylalkyl, arylalkenyl, arylalkynyl, heteroarylalkyl, heteroarylalkenyl, heteroarylalkynyl, heterocyclyl, heterocyclylalkyl, heterocyclylalkenyl and heterocyclylalkynyl, and each occurrence of R₄ is a metal coordinating group. In yet another embodiment, each occurrence of R₁ is independently C₄-C₈ alkyl. In yet another embodiment, each occurrence of R₁ is C₆H₁₃. In yet another embodiment, each occurrence of R₂ is hydrogen. In yet another embodiment, the metal coordinating group in each occurrence of R₄ is independently selected from the group consisting of thioether, pyridyl, bipyridyl, terpyridyl, anilino, amino, carboxylate, phosphate, sulfate, amido, sulfonamido, hydroxy, sulfhydryl, and cyano. In yet another embodiment, each occurrence of R₄ is independently SR₃, wherein each occurrence of R₃ is independently H or C₁-C₁₀ alkyl. In yet another embodiment, each occurrence of R₃ is independently H or C₁-C₅ alkyl. In yet another embodiment, each occurrence of R₃ is C₂H₅. In yet another embodiment, the cage molecule comprises COP-11, or any salt thereof.

In one embodiment, the composition further comprises nanoparticles, wherein the nanoparticles are bound to the cage molecule through the two or more metal coordinating groups. In yet another embodiment, the nanoparticles comprise a metal nanoparticle, magnetic nanoparticle, fluorescent nanoparticle, semiconductor nanoparticle, quantum dot nanoparticle, dielectric nanoparticle, or any combinations thereof. In yet another embodiment, the metal nanoparticles comprise gold, silver, aluminum, copper, nickel, iron, chromium, titanium, platinum, cobalt, palladium, or any combinations thereof.

### Methods

The invention includes a method of preparing a self-assembling nanocomposite structure on at least a fraction of the surface of a solid substrate. The method comprises the step of (a) providing a solid substrate. The method further comprises the step of (b) depositing a layer of nanoparticles on at least a fraction of the substrate surface. The method further comprises the step of (c) applying sequentially to the at least a fraction of the substrate surface a solution of a shape-persistent three-dimensional cage molecule and a solution of nanoparticles, wherein the cage molecule comprises two or more metal coordinating groups, whereby binding of the nanoparticles to the two or more metal coordinating groups allows for the self-assembly of the nanoparticles into a nanocomposite material. The method further comprises the step of (d) optionally removing excess solution from the at least a fraction of the substrate surface. The method further comprises the step of (e) repeating steps (c) and (d) until the desired thickness of the self-assembling nanocomposite structure on the substrate surface is obtained.

In one embodiment, the nanoparticles comprise a metal nanoparticle, magnetic nanoparticle, fluorescent nanoparticle, semiconductor nanoparticle, quantum dot nanoparticle, dielectric nanoparticle, or any combinations thereof. In another embodiment, step (b) comprises contacting the at least a fraction of the substrate surface with nanoparticles of opposite charge. In yet another embodiment, the at least a fraction of the substrate surface is rendered positively charged by amine functionalization. In yet another embodiment, the applying of step (c) comprises drop casting. In yet another embodiment, the cage molecule has a polyhedron shape selected from the group consisting of trigonal prismatic, tetrahedron, octahedron, cubic, dodecahedron, pentagonal prismatic, hexagonal prismatic, and any combinations thereof.

In one embodiment, the cage molecule is selected from the group consisting of formula (I), formula (II), any combinations thereof, and any salts thereof:

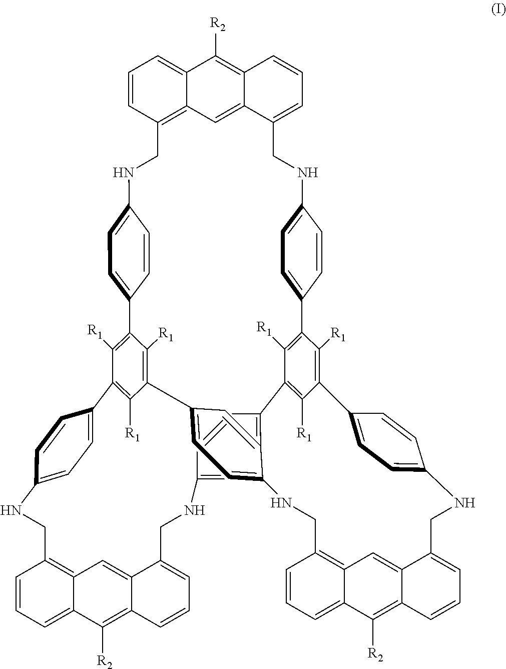

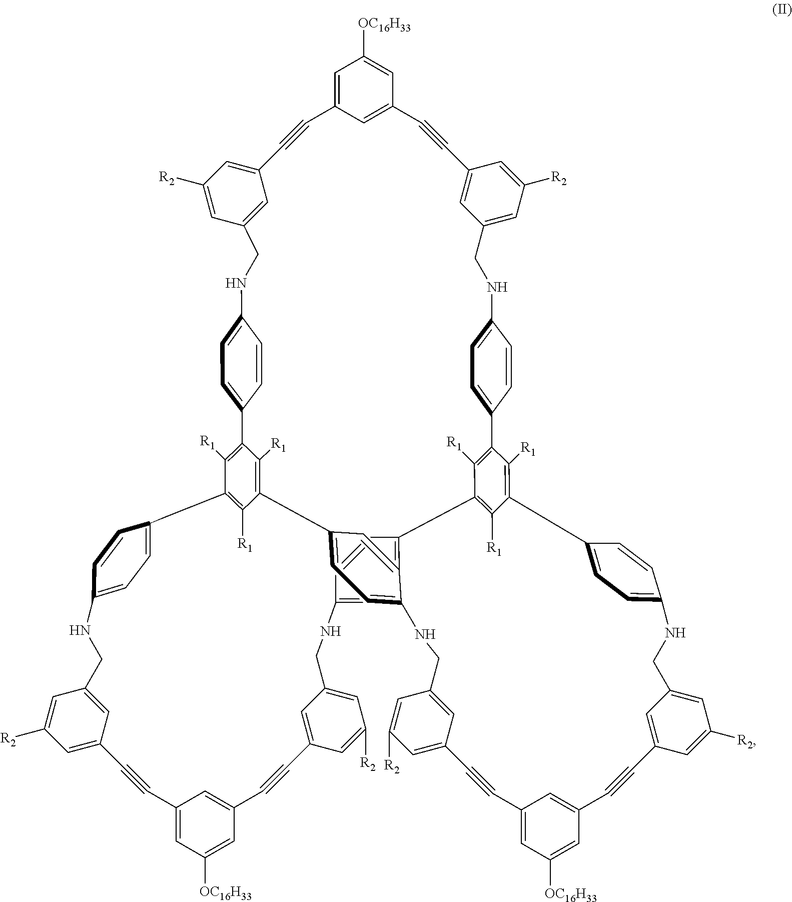

wherein each occurrence of R₁ is independently C₂-C₁₀ alkyl, and each occurrence of R₂ is independently selected from the group consisting of aryl, heteroaryl, arylalkyl, arylalkenyl, arylalkynyl, heteroarylalkyl, heteroarylalkenyl, heteroarylalkynyl, heterocyclyl, heterocyclylalkyl, heterocyclylalkenyl and heterocyclylalkynyl, further wherein R₂ comprises a metal coordinating group. In yet another embodiment, each occurrence of R₁ is independently C₄-C₈ alkyl. In yet another embodiment, each occurrence of R₁ is C₆H₁₃ (hexyl). In yet another embodiment, the metal coordinating group in each occurrence of R₂ is independently selected from the group consisting of pyridyl, bipyridyl, terpyridyl, anilino, amino, carboxylate, phosphate, sulfate, amido, sulfonamido, hydroxy, sulfhydryl, and cyano. In yet another embodiment, the metal coordinating group in each occurrence of R₂ is pyridyl. In yet another embodiment, each occurrence of R₂ is independently pyridin-4-yl or 2-(pyridin-4-yl)vinyl. In yet another embodiment, the cage molecule comprises COP-3P, COP-6VP, any salt thereof or any combinations thereof. In yet another embodiment, the metal nanoparticles comprises gold, silver, aluminum, copper, nickel, iron, chromium, titanium, platinum, cobalt, palladium, or any combinations thereof.

In one embodiment, the cage molecule is selected from the group consisting of formula (III), and any salts thereof:

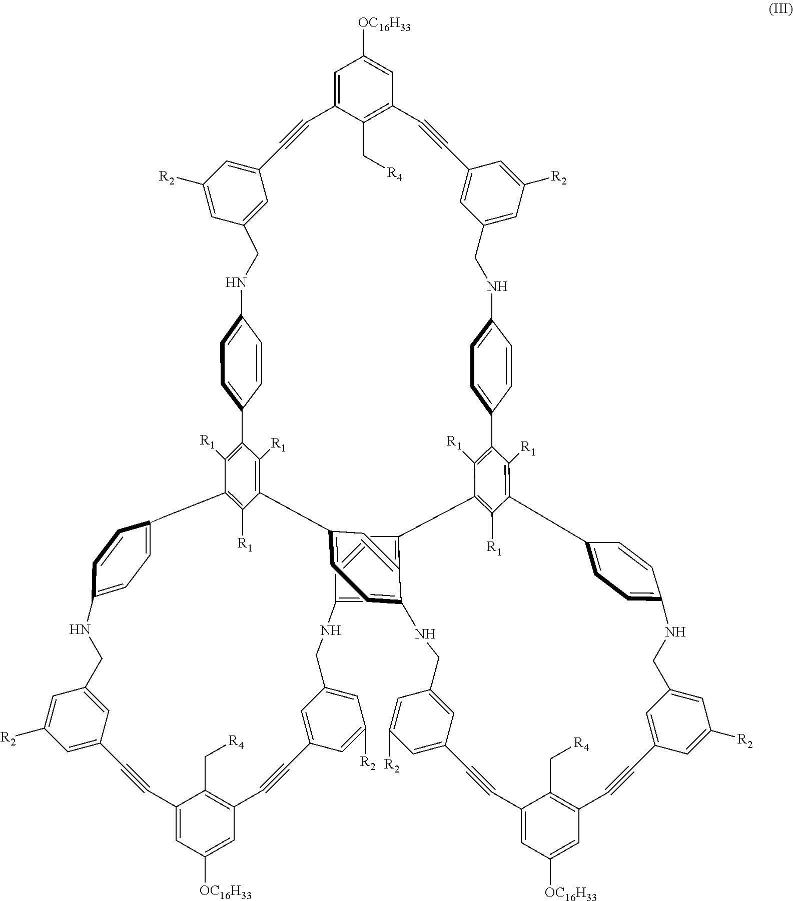

wherein for formula (III), each occurrence of R₁ is independently C₂-C₁₀ alkyl, each occurrence of R₂ is independently selected from the group consisting of hydrogen, aryl, heteroaryl, arylalkyl, arylalkenyl, arylalkynyl, heteroarylalkyl, heteroarylalkenyl, heteroarylalkynyl, heterocyclyl, heterocyclylalkyl, heterocyclylalkenyl and heterocyclylalkynyl, and each occurrence of R₄ is a metal coordinating group. In yet another embodiment, each occurrence of R₁ is independently C₄-C₈ alkyl. In yet another embodiment, each occurrence of R₁ is C₆H₁₃. In yet another embodiment, each occurrence of R₂ is hydrogen. In yet another embodiment, the metal coordinating group in each occurrence of R₄ is independently selected from the group consisting of thioether, pyridyl, bipyridyl, terpyridyl, anilino, amino, carboxylate, phosphate, sulfate, amido, sulfonamido, hydroxy, sulfhydryl, and cyano. In yet another embodiment, each occurrence of R₄ is independently SR₃, wherein each occurrence of R₃ is independently H or C₁-C₁₀ alkyl. In yet another embodiment, each occurrence of R₃ is independently H or C₁-C₅ alkyl. In yet another embodiment, each occurrence of R₃ is C₂H₅. In yet another embodiment, the cage molecule comprises COP-11, or any salt thereof.

In one embodiment, the metal nanoparticles comprise gold, silver, aluminum, copper, nickel, iron, chromium, titanium, platinum, cobalt, palladium, or any combinations thereof.

### Disclosure

In one aspect, the present invention relates to the discovery of a novel method of self-assembling nanomaterials without using a template. As demonstrated herein, it is possible to accomplish highly controlled self-assembly of nanoparticles by using binding molecules grafted on the nanoparticles. As opposed to the template-directed and liquid crystal based self-assembly methods (both of which rely on physical interactions between particles and template or matrix), the method of the invention takes advantage of chemical interactions of the binding molecules, potentially providing much greater degree of control in the self-assembly process.

Plasmonic nanoparticle self-assembly may be achieved using 3D shape-persistent cage molecules. Well-defined, rigid, 3D purely organic cage-like molecules have attracted great research attention due to their unique shape-persistency, structure-tunability, and chemical and thermal stability. These molecular cages have been utilized in various fields, such as carbon capture (Jin et al., 2010, Angew. Chem. Int. Ed. 49:6348-6351; Jin et al., 2011, J. Am. Chem. Soc. 133:6650-6658; Jin et al., 2012, Chem. Sci. 3:874-877), fullerene separation (Zhang et al., 2011, J. Am. Chem. Soc. 133:20995-21001; Zhang et al., 2012, Chem. Commun. 48:6172-6174), and light harvesting (Lohrman et al., 2012, Chem. Commun. 48:8377-8379).

The present disclosure is the first example of the use of rigid cage molecules as linkers to control the nanoparticle assembly. Recent significant progress in the development of dynamic covalent chemistry (DCC) enables the highly efficient synthesis of cage compounds in a modular fashion. The modular construction of cage molecules provides the design flexibility required to control the nanoparticle assembly process. Also, the shape-persistent nature of the molecules leads to highly stable and robust structures. The present invention contemplates all possible polyhedron shapes of cage molecules known to those skilled in the art, such as but not limited to trigonal prismatic, tetrahedron, octahedron, cubic, dodecahedron, pentagonal prismatic, and hexagonal prismatic.

As reported herein, successful self-assembly of gold nanoparticles was achieved using two distinct types of cage molecules as linkers. The results of the present disclosure shed light on how the molecular structures affect and thus can be used to control the self-assembly process.

### Synthesis and Self-Assembly

Two cage molecules, also called covalent organic polyhedrons (COP), namely COP-3P and COP-6VP, were designed, synthesized and investigated along with a small ligand molecule, TP, which was used as a reference compound. Study of these molecules provided insights into how the binding efficiency and kinetics are influenced by the cage structures on the molecular-level.

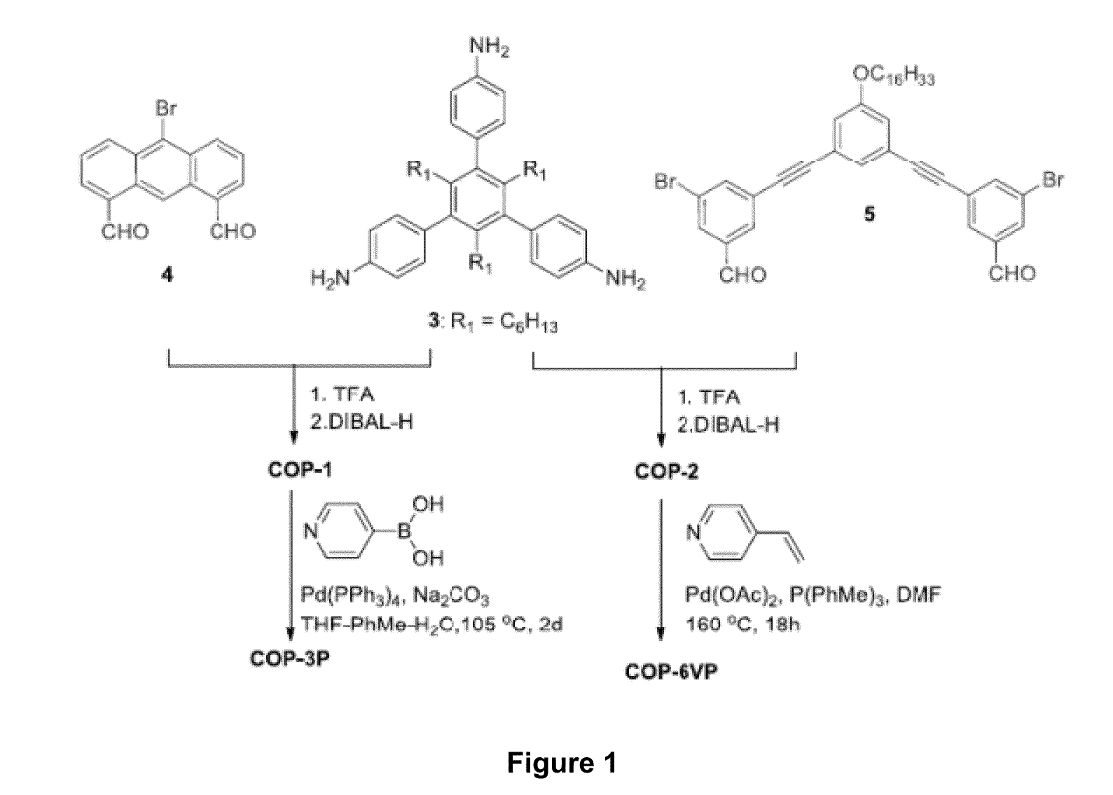

As illustrated in FIG. 1, pyridyl groups were installed on all three molecules as anchoring sites for binding with gold nanoparticles. TP and COP-3P have three pyridyl groups whereas COP-6VP has six. Conventionally, the organic cage compounds are prepared through kinetically controlled, irreversible coupling reactions, which generally provide cage products in very low yield due to the intrinsic “over-shooting” problem (Zhang & Moore, 2006, Angew. Chem. Int. Ed. 45:4416-4439). However, recent advances in dynamic covalent chemistry have enabled facile access to nanometer-sized covalently linked cage molecules from simple synthetic precursors (Jin et al., 2010, Angew. Chem. Int. Ed. 49:6348-6351; Zhang et al., 2011, J. Am. Chem. Soc. 133:20995-21001). Trigonal prismatic cage COP-1 and COP-2 were constructed through dynamic imine metathesis, in which the product formation was under thermodynamic control, from 2:3 equivalent of triamine and dialdehyde building units. Bromo functional groups were installed on each of the side arms as the synthetic handle for further introducing the nanoparticle binding sites. The pyridine binding sites were then installed through cross-coupling reaction of COP-1 and COP-2 with pyridine-4-boronic acid and 4-vinylpyridine respectively to provide COP-3P and COP-6VP.

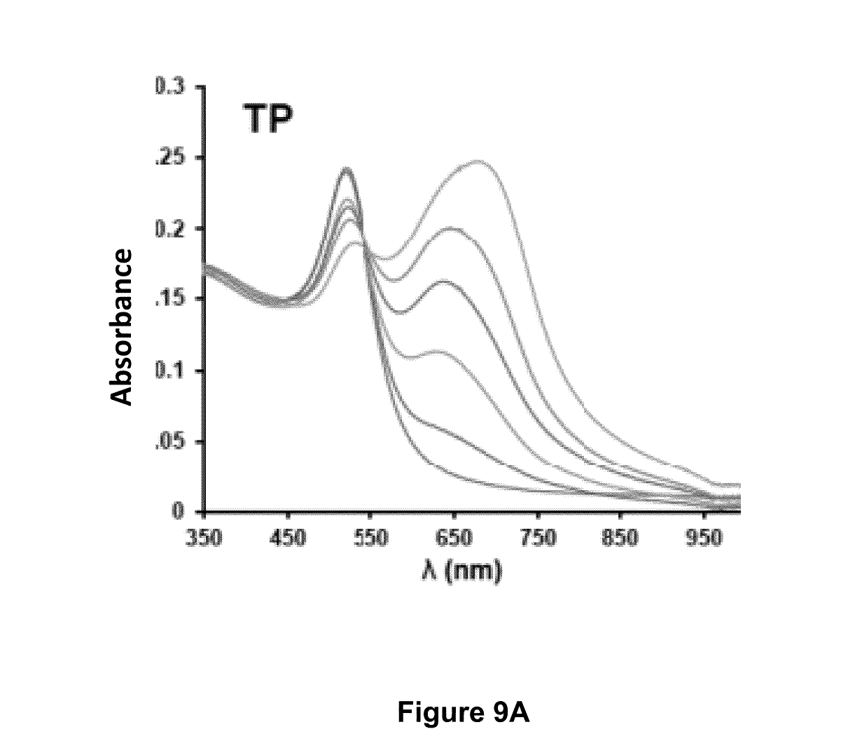

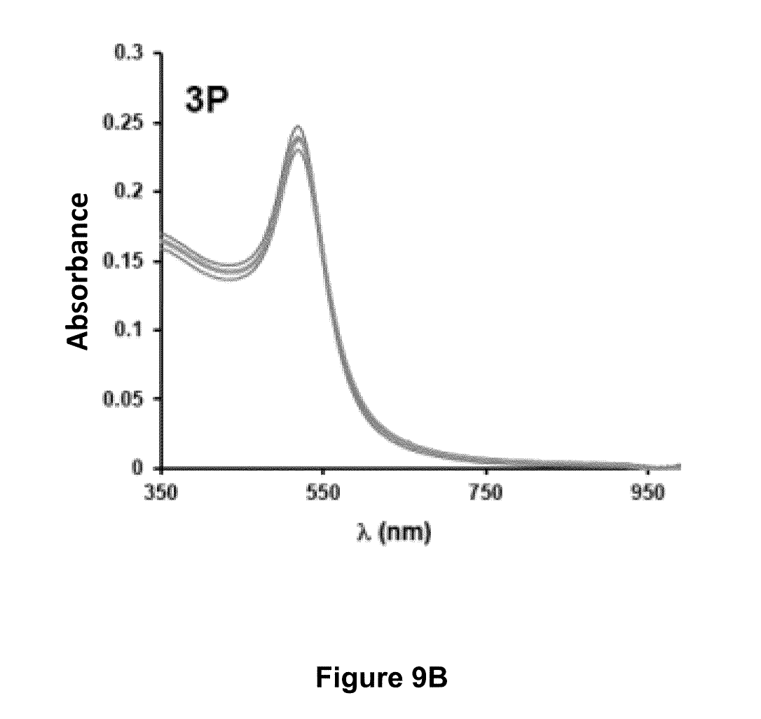

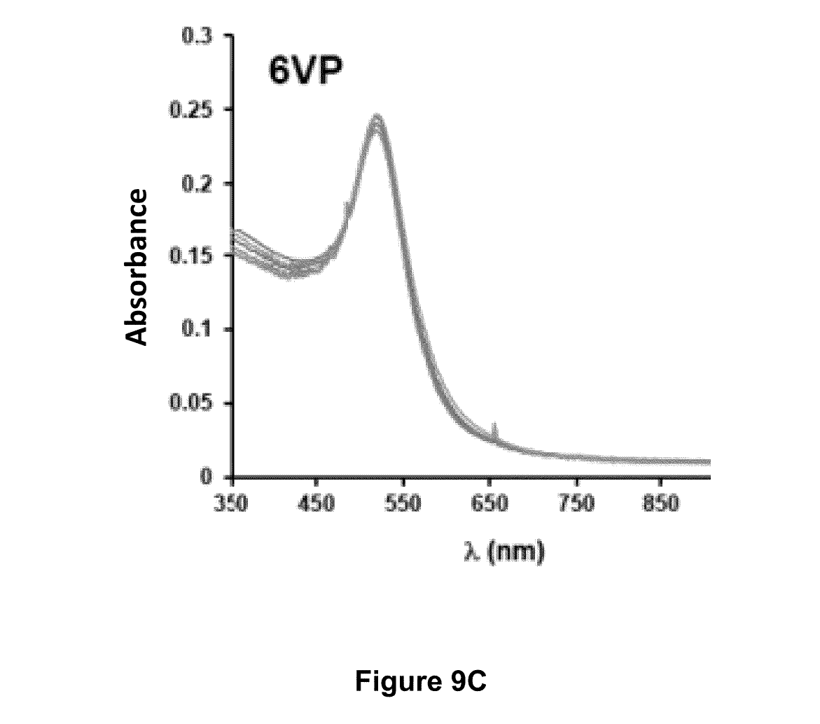

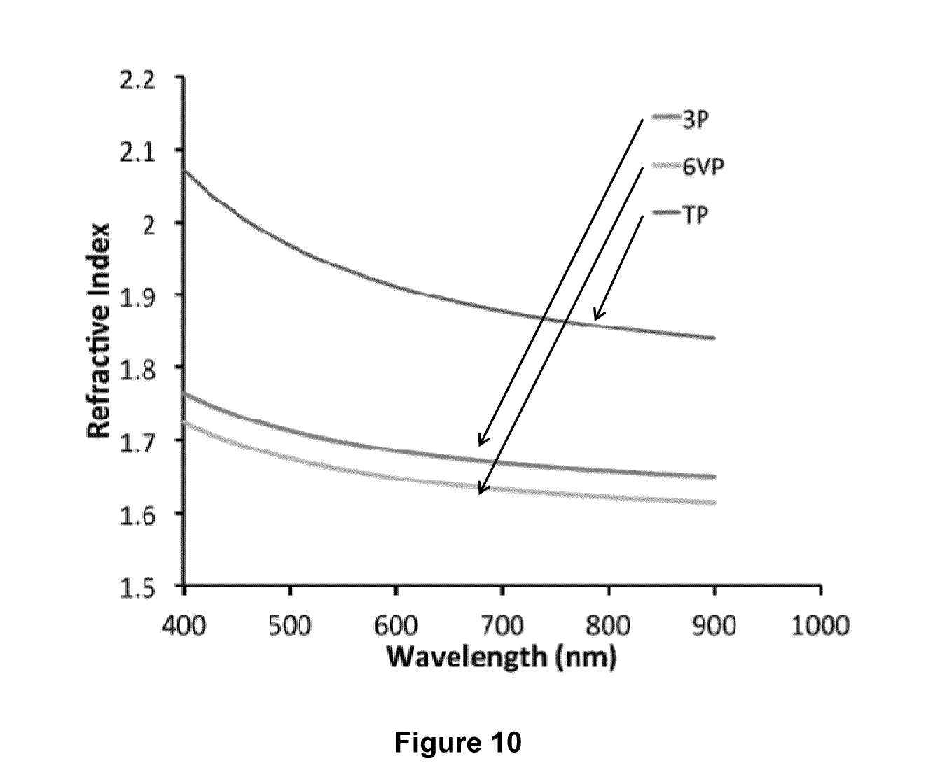

Thanks to its small size (14.7 Å), TP molecules can readily rotate and reorient themselves and thus bind with gold nanoparticles efficiently (Kaminker et al., 2010, Angew. Chem. Int. Ed. 49:1218-1221). The COP molecules are larger and therefore not as nimble as TP. This difference was obvious when the gold nanoparticle solution and cage molecule solution were mixed for a quick test of binding kinetics. The original gold nanoparticle solution was red due to the strong surface plasmon absorption peak at 520 nm. When TP was added, the solution immediately turned purple. The optical extinction spectrum revealed the emergence of a second peak near 700 nm, which was due to the coupling between individual nanoparticle plasmon resonances. This observation was direct evidence of nanoparticle cluster formation and suggests highly efficient binding between TP ligand molecules and gold nanoparticles. For both COP-3P and COP-6VP, the solution color change occurred slowly over an hour or longer, indicating much slower binding kinetics. Furthermore, in contrast to TP, mixing gold nanoparticles with COP-3P or COP-6VP led only to a slight red shift of absorption peak without showing the second peak near 700 nm on the UV-Vis spectrum (FIG. 9). These results were consistent with those from self-assembly.

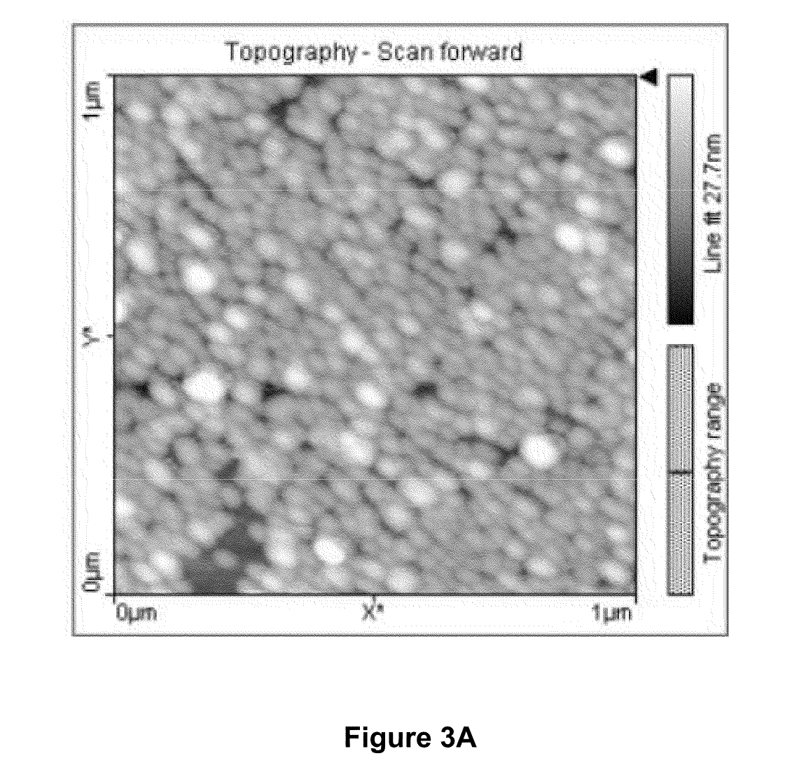

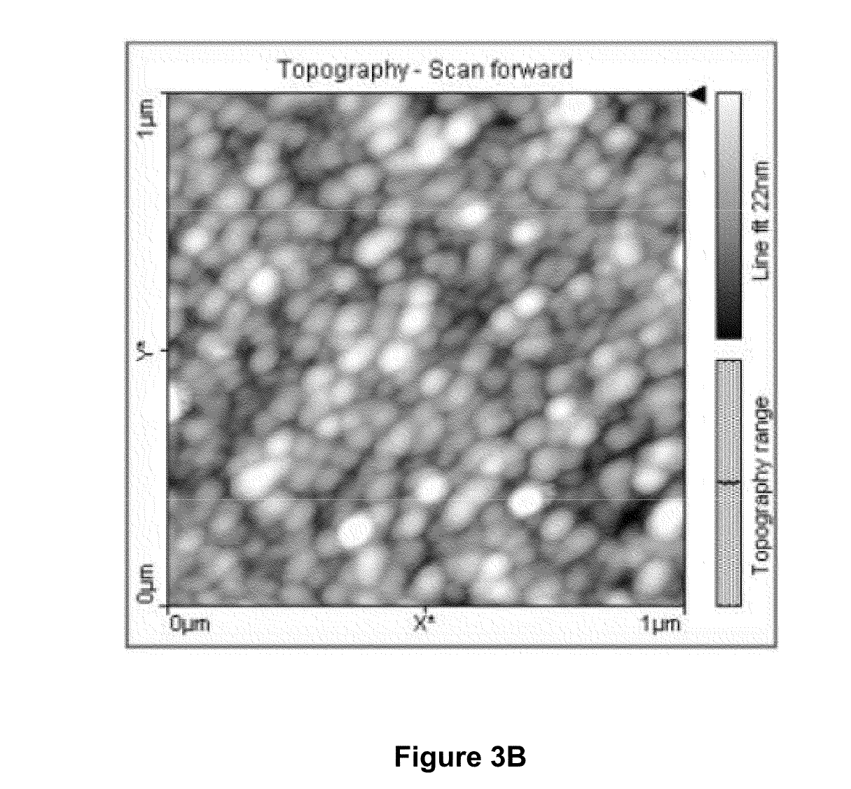

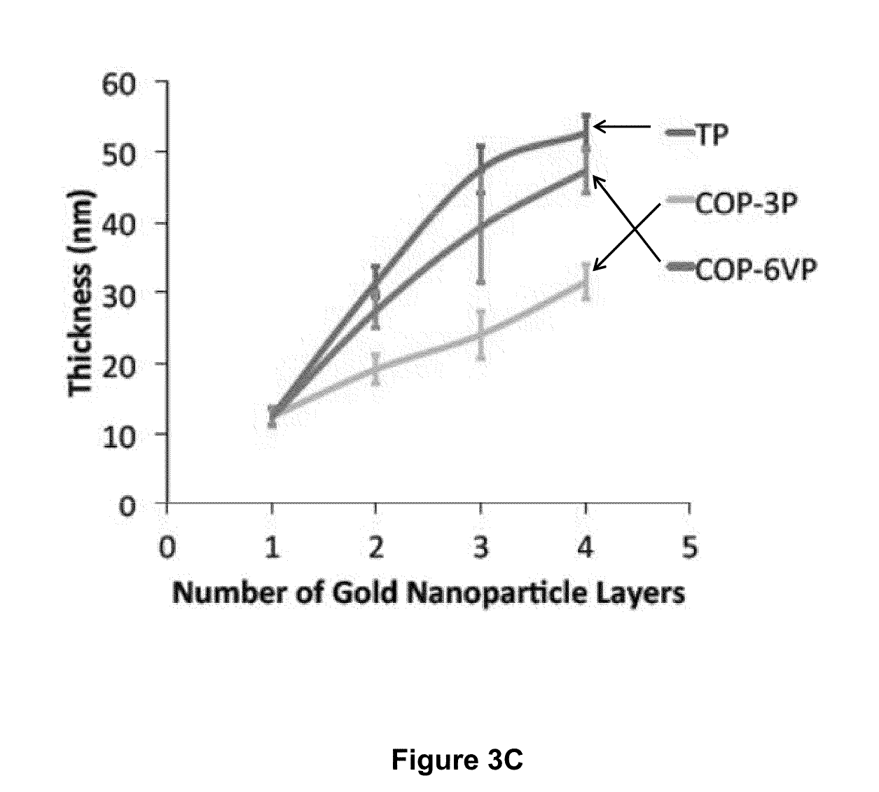

Self-assembly of gold nanoparticles were conducted in a layer-by-layer fashion. In this process, the first layer of gold nanoparticles on the substrate was formed by electrostatic interaction between the negatively charged gold nanoparticles in aqueous solution and the substrate surface positively charged by amine functionalization. Atomic force microscopy (AFM) showed that a monolayer of high-density, well-dispersed gold nanoparticle was formed (FIG. 3A). The particle density was measured to be ˜1000 μm⁻², which was comparable to what has recently been reported for gold nanoparticle deposition via electrostatic interaction (Cunningham et al., 2011, J. Phys. Chem. C 115:8955-8960). The average center-to-center spacing between two neighboring nanoparticles was 28 nm. On top of the monolayer prepared this way, cage molecule solution and gold nanoparticle solution were applied sequentially by drop casting method. After each cycle of sequential application of cage molecule solution and gold nanoparticle solution to the self-assembly surface, the surface topography and layer thickness were measured by AFM. The AFM images generally showed a surface with well dispersed gold nanoparticles, as shown in the example of four layer gold nanoparticles self-assembled with COP-3P molecules (FIG. 3B). The surfaces of TP and COP-6VP mediated self-assembly exhibited similar topographies (results not shown). As the number of layers of self-assembled nanoparticles was increased, the average particle size increased to 45-55 nm in diameter, indicating a small degree of clustering of nanoparticles on the self-assembly surface.

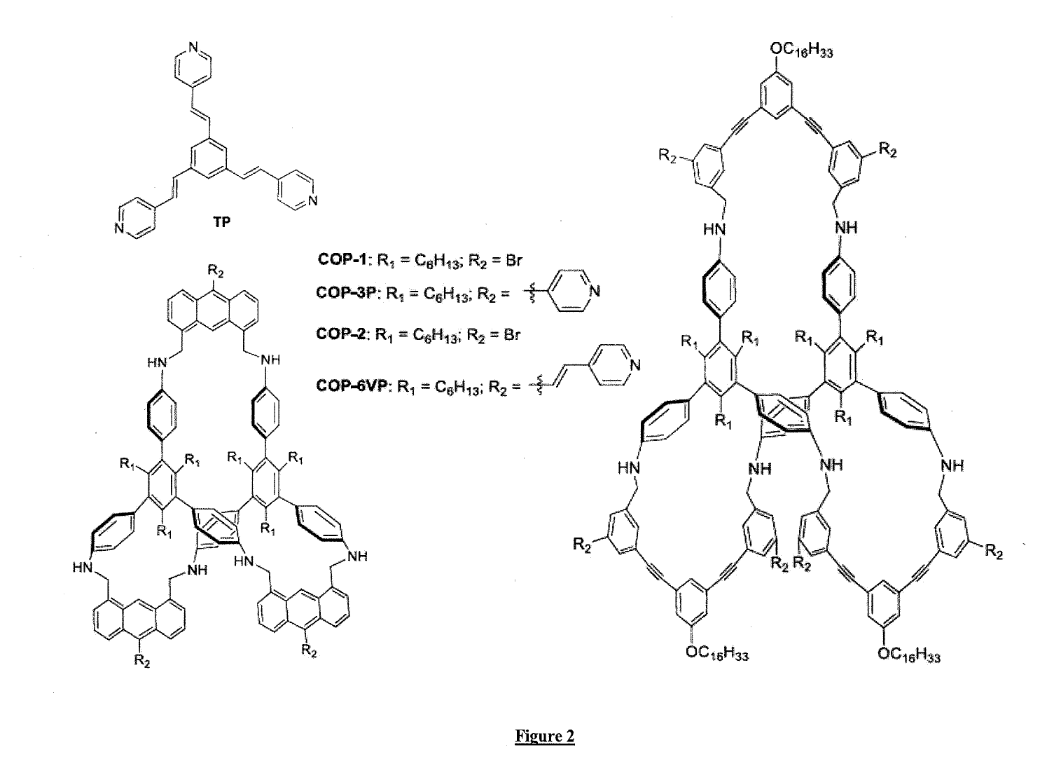

To determine whether the gold nanoparticle layers were indeed assembled in layer-by-layer fashion, thickness was measured by AFM as the self-assembly was being carried out. As illustrated in FIG. 3C, the total gold nanoparticle layer thickness increased in all cases as more cycles of self-assembly were conducted, indicating that the gold nanoparticles were captured and bound to the growth surface by the linker molecules used for self-assembly. However, the thickness was substantially lower for the samples self-assembled with COP-3P molecules than other molecules. This was attributed to the less efficient binding between COP-3P and gold nanoparticles, which was expected from the molecular structure of COP-3P. As illustrated in FIG. 2, COP-3P had pyridyl binding groups rigidly attached to the cage frame. In contrast, COP-6VP had more flexible vinyl groups (through vinyl and phenyl C—C single bond rotation) linking the pyridyls to the cage frame, thereby providing rotational freedom to the binding groups. As a result, TP and COP-6VP molecules with flexible binding groups could bind more efficiently with gold nanoparticles than COP-3P molecule.

In the thickness data, the less efficient binding of COP-3P with gold nanoparticles manifested itself with lower thicknesses. For the other two molecules, TP and COP-6VP, the thicknesses were similar. The differences in thicknesses were 5-8 nm, which was substantially smaller than the single gold nanoparticle size, 14 nm. It could thus be stated that highly efficient binding with gold nanoparticles was achieved in both cases. While the thickness differences were small, the thickness was consistently larger with TP than COP-6VP. This result showed that there was some correlation between the molecule size and binding efficiency. TP was the smallest planer molecule and thus able to readily diffuse, rotate and reorient itself to bind with gold nanoparticles. This allowed high accessibility of ligand group to nanoparticles, leading a denser nanoparticle array on the surface. The larger size COP-6VP molecule was not as nimble as TP and consequently exhibited less efficient binding, which resulted in lower thicknesses than TP. However, despite the substantially larger size, COP-6VP resulted in thicknesses only slightly smaller than TP. This was because COP-6VP had a larger number (six) of binding groups than TP, which had three.

### Characterization and Analysis

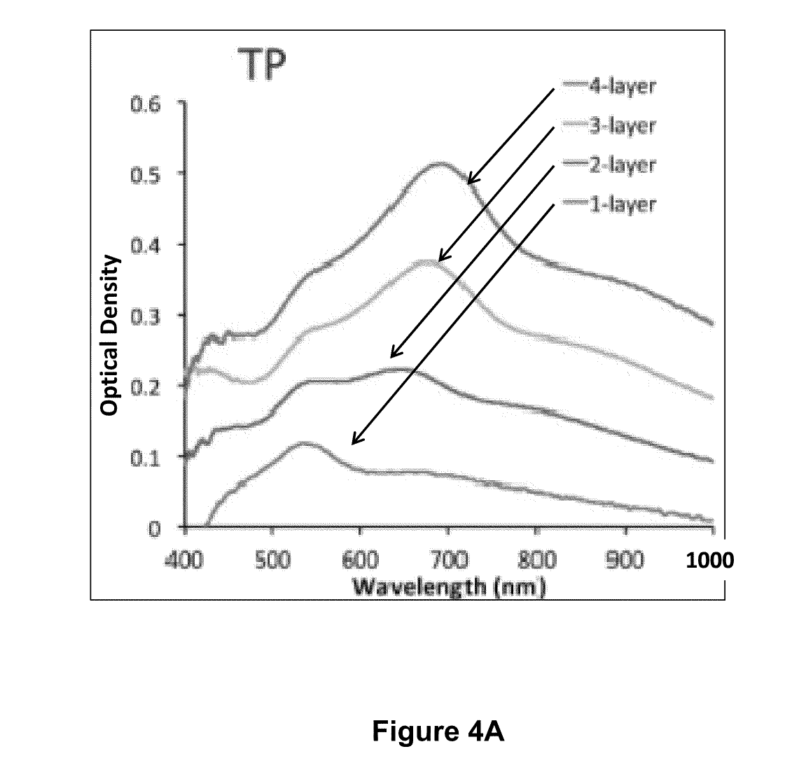

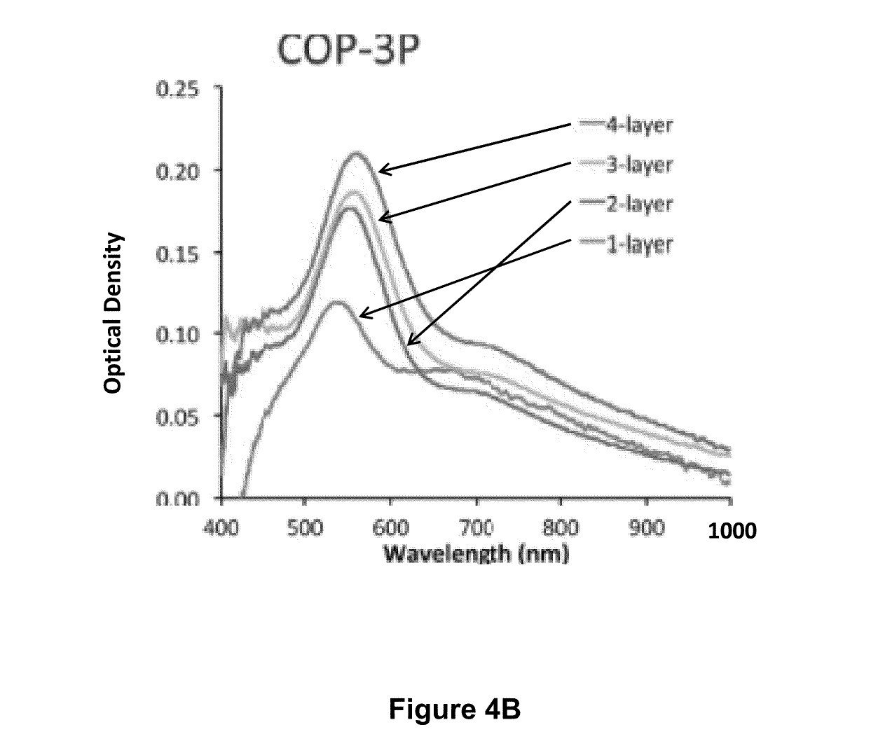

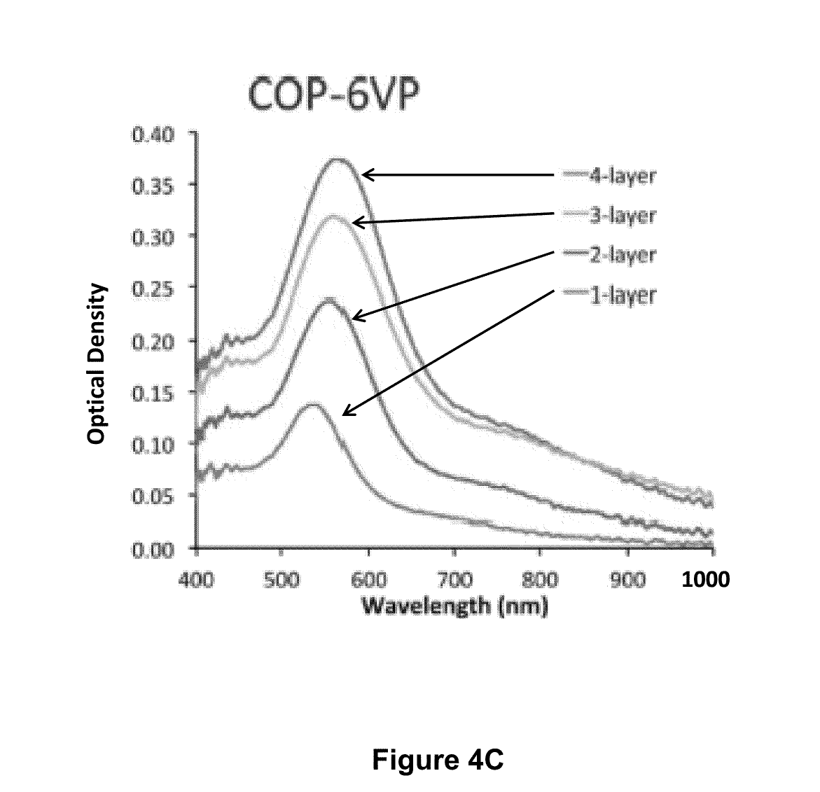

Optical extinction spectroscopy was carried out for the self-assembled layers of gold nanoparticles. FIG. 4 illustrates the extinction spectra of 1-4 layers of gold nanoparticles self-assembled with TP, COP-3P, and COP-6VP molecules. In all cases, the extinction increased as more layers were assembled. This was consistent with the thickness data, which showed the thickness increased with increasing number of self-assembled layers with all three molecules. The peak extinction values showed significant differences depending on which molecule was used for self-assembly. TP resulted in the highest extinction and COP-3P gave the lowest, with COP-6VP showing slightly lower extinction than TP. Since none of the linker molecules has absorption above 400 nm, the optical extinction in the visible spectrum should be proportional to the total number of gold nanoparticles present in the light path, which in the samples is determined by the thickness of the self-assembled layer and density of gold nanoparticles in it. The peak extinction observed in TP, COP-3P and COP-6VP samples was in excellent agreement with the thickness data obtained by AFM, once again confirming that TP led to the most efficient binding, followed by COP-6VP and COP-3P in that order.

The optical extinction spectra offered another piece of information on gold nanoparticle packing from the position of peak extinction. The origin of the extinction peak is obviously the surface plasmon resonance of gold nanoparticles. According to the Mie theory (Bohren & Huffman, 2004, Absorption and Scattering of Light by Small Particles; WILEY-VCH Verlag GmbH & Co. KgaA: Weinheim), a 14 nm size gold sphere placed on a glass substrate should exhibit extinction peak at around 530 nm, which was observed from the monolayer samples (FIG. 4). This result suggests that the gold nanoparticles are uniformly distributed on the surface with minimal aggregations. As more gold nanoparticle layers were added by subsequent self-assembly processes, the surface plasmon resonances of individual gold nanoparticles couple together with those of neighboring nanoparticles, and the resultant delocalization consequently leads to the lowering of resonance frequency and red shift in the optical extinction peak. The peak shift depended on the inter-particle spacing. The closer the nanoparticles, the stronger the coupling between the neighboring particles, and thus the larger the red shift in optical spectrum. This well-known behavior also observed in dimers (Atay et al., 2004, Nano Lett. 4:1627-1631) was consistent with the effective medium analysis presented elsewhere herein. In the optical extinction spectra presented in FIG. 4, TP showed by far the largest red shift. In fact, the TP spectra in FIG. 4A indicated that the original extinction peak at 530 nm, dominant in the monolayer sample, became overwhelmed by the fast emerging second peak at a longer wavelength. The position of the second peak exhibited a modest red shift from 640 nm for 2 layer sample to 685 nm for 4 layer sample. In contrast, the COP-3P spectra in FIG. 4B showed a very small red shift with increasing number of layers to 550 nm for the 4 layer sample. In the COP-6VP spectra, the red shift was slightly larger, reaching 560 nm for the 4 layer sample (FIG. 4C). These observation suggests that the self-assembly with TP resulted in the most dense packing of gold nanoparticles with smallest inter-particle spacing, COP-3P produced the lowest density gold nanoparticle assembly, and COP-6VP slightly higher packing density.

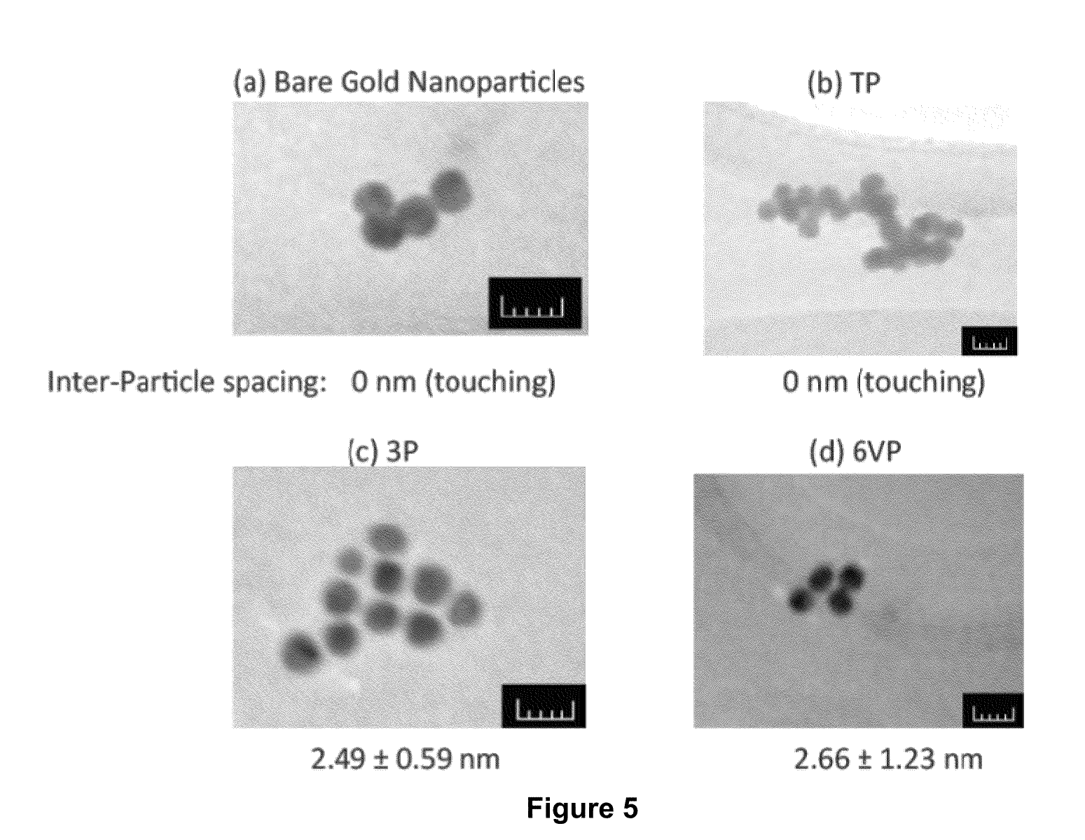

For more quantitative analysis, the extended Maxwell Garnett effective medium theory (Doyle, 1989, Phys. Rev. B 39:9852-9858; Yannopapas & Moroz, 2005, J. Phys. Condens. Matter 17:3717-3734) was used to fit the optical extinction spectra. In this method, the polarizability of individual gold nanoparticle was first calculated by using the Mie theory, and then the Maxwell Garnett mixing rule (Maxwell Garnett, 1904, Phil. Trans. R. Soc. Lond. A 203:385-420) was used to calculate the complex effective refractive index for the composite structure. The extended Maxwell Garnett effective medium theory provides adequate descriptions of metal nanoparticle clusters, as evidenced by comparisons with the rigorous numerical modeling by the multiple scattering method (Tamma et al., 2010, Appl. Opt. 49:A11-A17). In order to apply the extended Maxwell Garnett theory, the gold nanoparticle size, their density inside the self-assembled film and the refractive indexes of gold and the background medium needed to be determined. The average size of the gold nanoparticle was measured to be 14 nm by the scanning electron micrographs (SEMs). The average spacing between the nanoparticles is determined by the cage molecule and was directly measured by a series of transmission electron micrographs (TEMs). The TEM images clearly showed the interparticle spacing was related to the cage molecules used (FIG. 5). The bare nanoparticles and TP grafted nanoparticles formed clusters of touching nanoparticles, whereas the cage molecule coated nanoparticles showed finite gaps between them. The average spacing between the adjacent nanoparticles was determined by averaging 30-40 inter-particle spacings extracted from a series of TEM images. For COP-3P, the gap was measured to be 2.49±0.59 nm and for COP-6VP it was 2.66±1.23 nm. These values are in excellent agreement with the cage molecule sizes. From the molecular structure, the transverse size of COP-3P defined as the distance between adjacent binding sites was estimated to be 2.73 nm. For COP-6VP, since there were six binding sites, the distance between a pair of biding sites varied from 1.5 nm to 3.5 nm. Therefore, the slightly larger average gap size together with larger standard deviation obtained for COP-6VP was considered consistent with the molecular structure. It thus follows that the 3D structure of cage molecules is an important factor in creating finite spacing between nanoparticles.

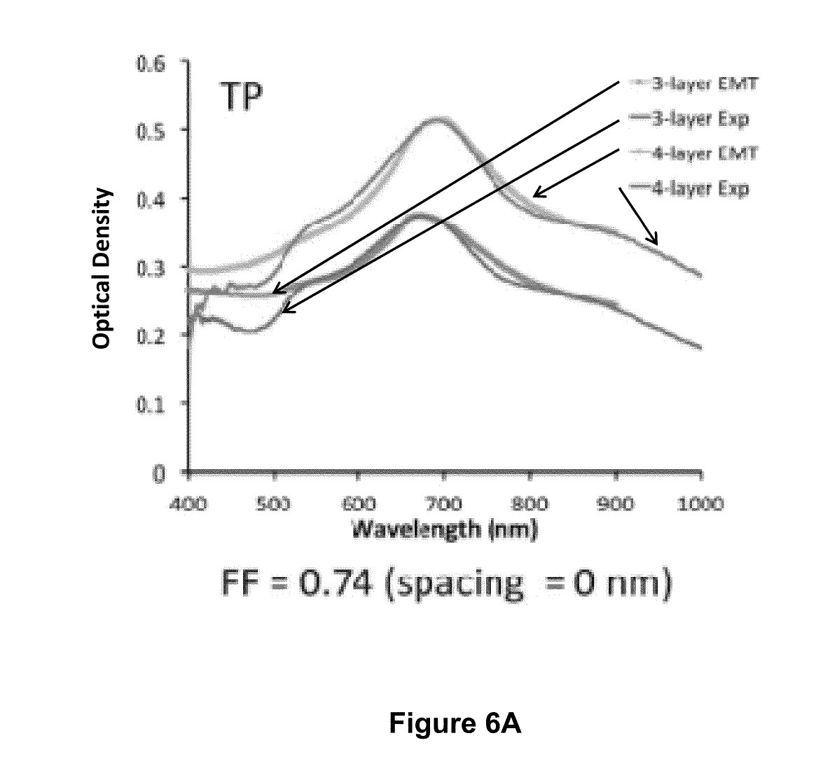

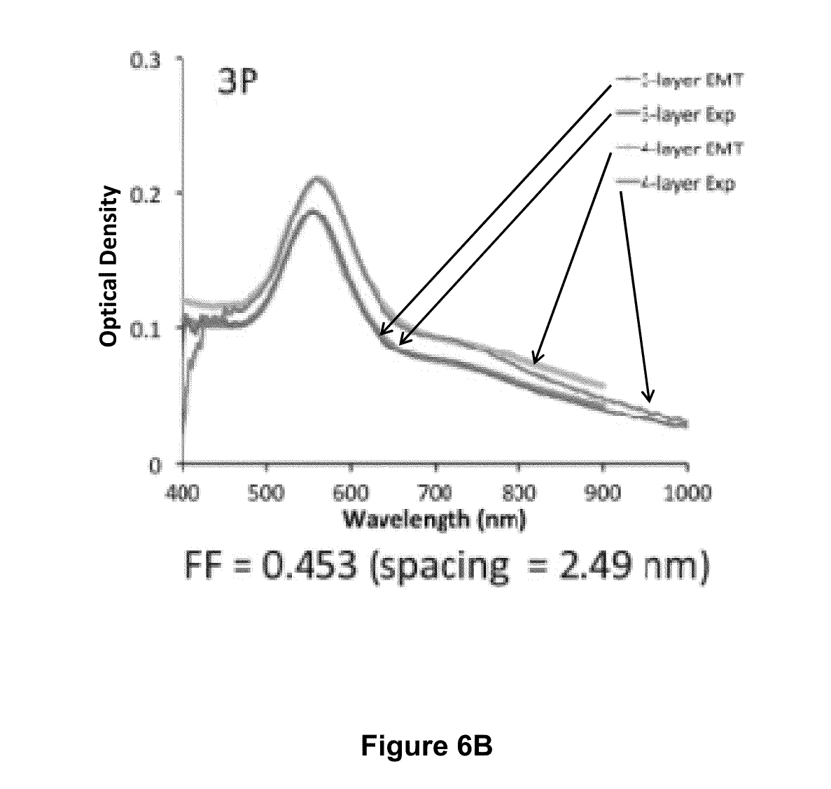

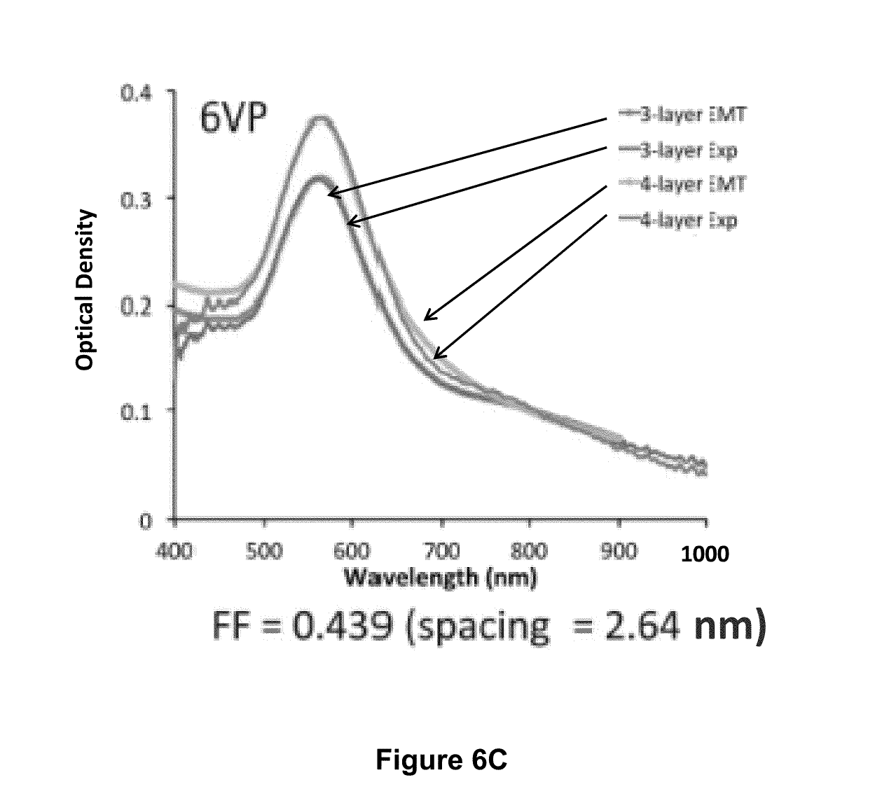

For the refractive index of gold, the experimentally measured dielectric function (Johnson & Christy, 1972, Phys. Rev. B 6:4370-4379) was used, but the imaginary part was increased to account for higher loss due to increased scattering in nanoscale geometry, as customarily done in plasmonic nanostructure research. The background medium refers to the space between gold nanoparticles and is composed of cage molecules and air. It was not possible to accurately determine the amount of cage molecules in the space between gold nanoparticles. The refractive index of cage molecule thin films was thus measured by ellipsometry, and the volume fraction of cage molecules in the background medium was then used as a fitting parameter for the experimentally measured optical extinction spectra. In all six cases presented in FIG. 6, excellent fitting with cage molecule volume fraction of 0.4-0.5 in the background medium was obtained. The metamaterial structures self-assembled with COP-3P and COP-6VP molecules exhibited a small feature in the long wavelength region between 700 and 800 nm. This may be due to the formation of larger clusters of gold nanoparticles during the drying process introduced between application of cage molecule solution and gold nanoparticle solution. When the samples were kept wet at all times, this long wavelength feature disappeared.

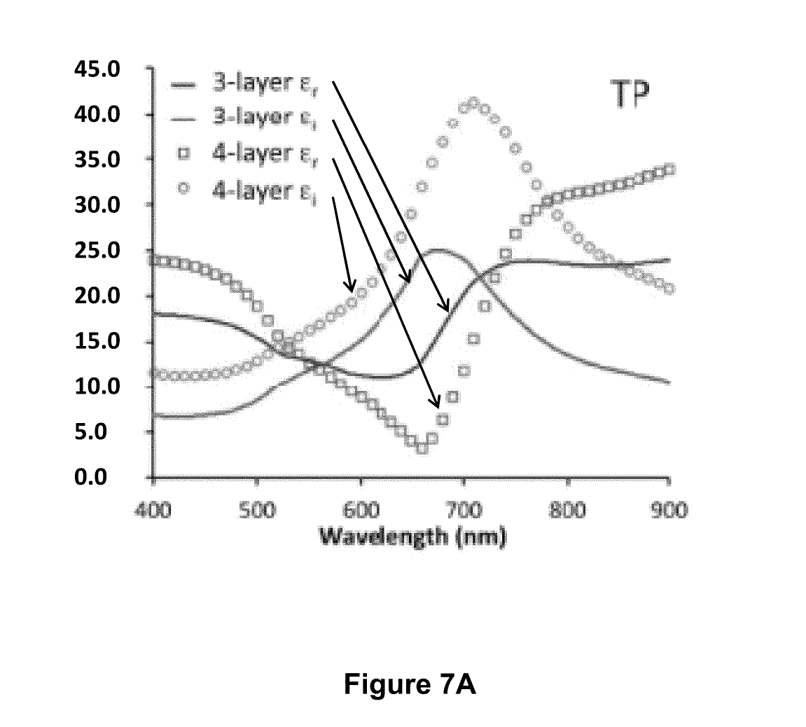

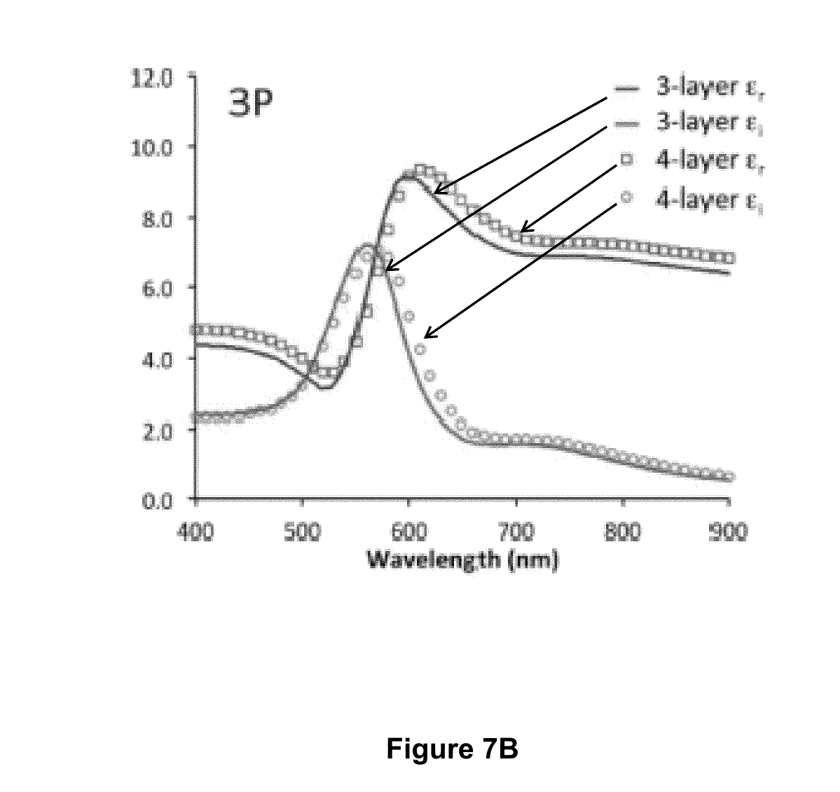

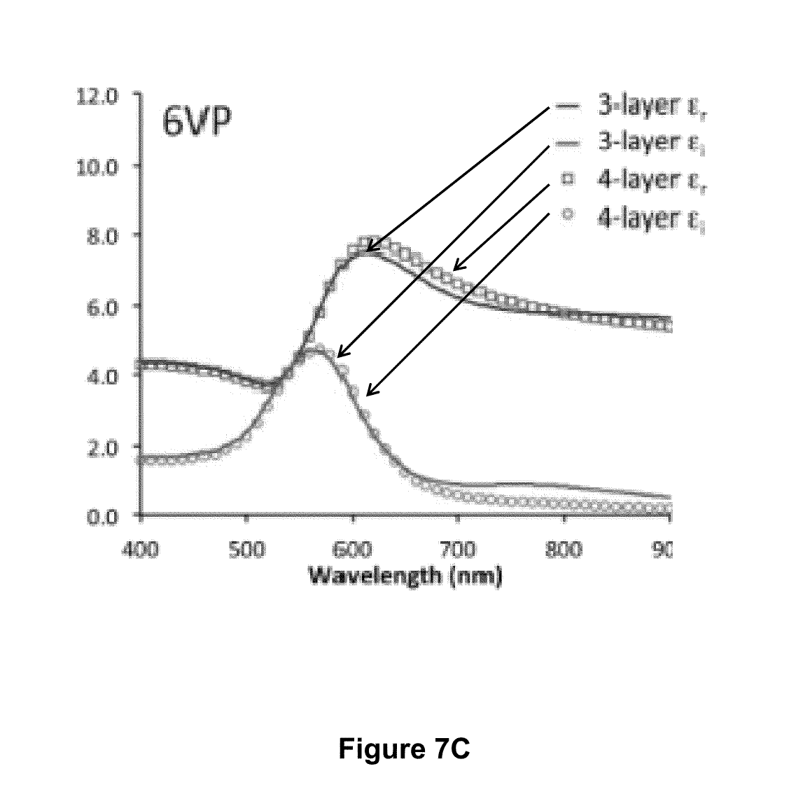

From the fitting by the effective medium theory presented in FIG. 6, effective permittivity can be extracted. The real and imaginary parts of effective permittivity are plotted in FIG. 7. The general features of the effective permittivity were similar in all three cases and in agreement with the classical oscillator model, in that the imaginary part exhibited a peak corresponding to the absorption band observed in experiments while the real part showed a wiggle around the resonance. The origin of the resonance was the surface plasmon resonance of individual gold nanoparticles shifted and broadened through the dipole-dipole interaction taken into account in the extended Maxwell Garnett effective medium theory. While the general behavior was the same for all three samples, a key difference was observed between TP and the two cage molecules. The two samples prepared with COP-3P and COP-6VP showed almost identical dielectric functions for the 3-layer and 4-layer cases while there were large discrepancies in TP. Ideally, the same dielectric function should be retrieved irrespective of the sample thickness if the effective medium theory is truly valid. The validity of various effective medium theories is well documented (see, for example, Milton, 2004, Theory of Composites, Cambridge University Press). Maxwell Garnett theory or extended Maxwell Garnett theory are generally considered better suited than other approaches such as Bruggeman theory, for the particulate composites (Abeles & Gittleman, 1976, Appl. Opt. 15:2328-2332; Yannopapas et al., 1999, Phys. Rev. B 60:5359-5365). Rigorously speaking, the Maxwell Garnett theory should be valid only in the small particle size and small volume fraction limit. However, for small particles, it was shown to be reasonably accurate over a surprising wide range of volume fraction (Tamma et al., 2010, Appl. Opt. 49:A11-A17), as confirmed by the present study. In the cases of COP-3P and COP-6VP samples, the fact that almost identical dielectric functions were retrieved for two distinct thicknesses suggested that meaningful dielectric functions were obtained. The differences between 3-layer and 4-layer cases were only a few percent at most wavelengths and less than 10% even at maximum. The small differences most likely stemmed from the small difference in the background index used to obtain the best fit to the experimental extinction spectra. Since the background index depends on the volume fraction of cage molecules within the gap space between the gold nanoparticles, there could exist small variations from sample to sample. The large discrepancies between the 3-layer and 4-layer dielectric functions obtained for TP, however, indicated the failure of the Maxwell Garnett effective medium theory. This was in fact expected because the Maxwell Garnett theory assumes particulate inclusions in a host medium and is well known to fail to provide the same result when the inclusion and host media are interchanged. Therefore, when the composite structure exhibits an interconnected network topology such as the one exhibited by the touching gold nanoparticles in TP samples, the Maxwell Garnett theory is not suitable. This was confirmed by the failure to obtain the same dielectric function for both the 3-layer and 4-layer samples self-assembled by TP. If the sample was composed of air “particles” in gold matrix, it would have been possible to use Maxwell Garnett theory by calculating the polarizability of air spheres in gold. However, in the present case, both gold and air were expected to exhibit network topology and thus the Maxwell Garnett approach was considered simply not applicable.

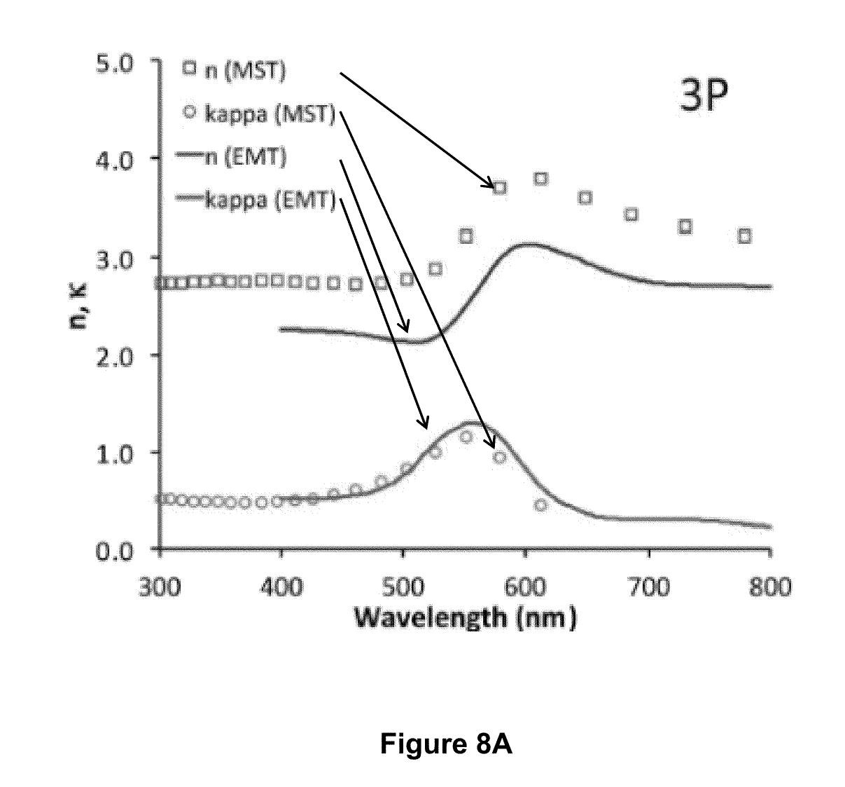

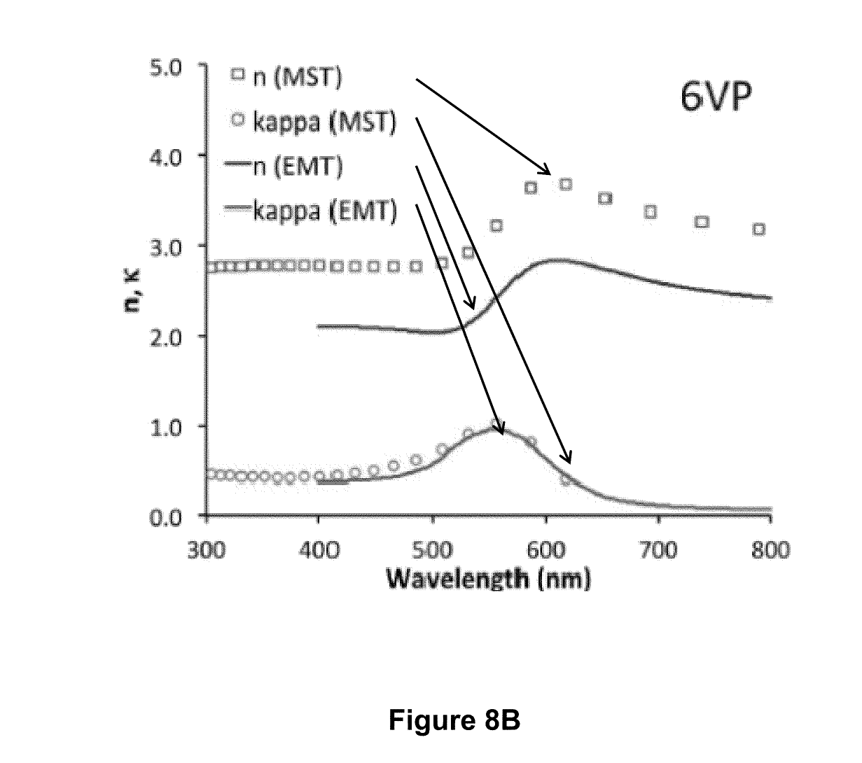

As further evidence that the retrieved dielectric functions for COP-3P and COP-6VP samples are meaningful, photonic band structure calculations were conducted using the multiple scattering theory (Stefanou et al., 1992, J. Phys.: Condens. Matter 4:7389-7400; Stefanou et al., 2000, Comp. Phys. Comm. 132:189-196). This is a rigorous approach without any approximation, other than the fact that the spherical wave expansion used to calculate the multiple scattering between spherical inclusions has to be terminated at a finite number, and is therefore a good test for the validity of effective medium approximation. Since the multiple scattering calculation provides the photonic band structure, or the ω-k relationship, the refractive index may be extracted from it and compared with the refractive indexes calculated from the effective permittivity given in FIG. 7. The real and imaginary parts of the refractive index calculated by the multiple scattering theory and effective medium theory are illustrated in FIG. 8. The extinction coefficient, κ, showed excellent agreement for both COP-3P and COP-6VP samples. For the real part of the refractive index, the effective medium results were slightly smaller than the multiple scattering results, but the spectral features such as the wavelengths of local maximum and minimum were in excellent agreement. There are two possible reasons for the observed differences. First, the Maxwell Garnett theory was known to underestimate the effective refractive index compared to the rigorous multiple scattering theory (Moroz & Sommers, 1999, J. Phys.: Condens. Matter 11:977-1008). The second reason is that the multiple scattering theory assumed perfectly periodic structures, whereas the real structures used for experiments and subsequently for fitting by the effective medium theory are random composites. In the present calculations, face-centered cubic arrangements of gold nanoparticles with interparticle spacing of 2.49 nm for COP-3P and 2.64 nm for COP-6VP samples were assumed, as determined by the TEM images and subsequently used for the effective medium calculations. It is therefore expected that the multiple scattering results would not precisely predict the experimental data and consequently would not agree perfectly with the effective medium results obtained by fitting the experimental data. The degree of disagreement would depend on various aspects of the composite structure. Given that gold nanoparticle would strongly interact with light due to surface plasmon resonance, the observed difference of 20-25% in refractive index between the multiple scattering theory and effective medium theory is considered reasonable. Multiple scattering calculations for the TP samples were not conducted because the numerical instability prevented simulating touching spheres, and also because the effective medium description itself was in question as discussed before. As discussed in the present disclosure, the experimentally measured optical extinction spectra could be fitted very well with the effective medium theory using the inter-particle spacing measured by TEM. Further, the effective medium results were reliable. From this, accurate control the interparticle spacing in the self-assembled film by the choice of cage molecules was evidenced. Inter-particle spacing is a critical parameter that dictates the coupling between the plasmon resonances and thus ultimately determines the effective dielectric function of the self-assembled structure. The capability of controlling the inter-particle spacing down to the nanometer scale affords us the ability to finely control the dielectric function of the self-assembled metamaterial structure.

The results reported herein demonstrate nanoparticle self-assembly enabled by shape-persistent 3D cage molecules. The modular construction of cage molecules allows for a precise control of inter-particle spacing down to the molecular level. Furthermore, the ability to change the number and flexibility of binding sites provides a means to tune the self-assembly process. In a non-limiting example, two distinct types of cage molecules were used to demonstrate successful self-assembly of gold nanoparticles. The experimentally observed self-assembly dynamics were consistent with the expectations from the molecular structures. A systematic and thorough analysis of the optical and structural characterization results showed that high-quality self-assembled metamaterial structures were obtained and their optical properties were explained well by the effective medium theory. Application of the effective medium theory indicated that the interparticle spacing in the self-assembled metamaterial structure was precisely controlled by the size of the cage molecule used in self-assembly. This results showed that the new self-assembly approach based on molecular cages could provide nanometric control over the self-assembled structure.

Every formulation or combination of components described or exemplified can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomers and enantiomer of the compound described individual or in any combination.

Although the description herein contains many embodiments, these should not be construed as limiting the scope of the invention but as merely providing illustrations of some of the presently preferred embodiments of the invention.

All references throughout this application (for example, patent documents including issued or granted patents or equivalents; patent application publications; and non-patent literature documents or other source material) are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application. In general the terms and phrases used herein have their art-recognized meaning, which can be found by reference to standard texts, journal references and contexts known to those skilled in the art. Any preceding definitions are provided to clarify their specific use in the context of the invention.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials and General Synthetic Methods

Reagents and solvents were purchased from commercial suppliers and used without further purification, unless otherwise indicated. Ether, tetrahydrofuran, toluene, CH₂Cl₂ and DMF were purified by solvent purification systems. All reactions, except those performed in aqueous solvent, were conducted under dry nitrogen in oven-dried glassware. Unless otherwise specified, solvents were evaporated using a rotary evaporator after workup. Flash column chromatography was performed by using a 100-150 times weight excess of flash silica gel 32-63 μm. Fractions were analyzed by TLC using TLC silica gel precoated-plates.

NMR spectra were taken on Inova 400 and Inova 500 spectrometers. CHCl₃ (7.26 ppm) was used as internal references in ¹H NMR, and CHCl₃ (77.23 ppm) for ¹³C NMR. ¹H NMR data were reported in order: chemical shift, multiplicity (s, singlet; d, doublet; t, triplet; q, quartet; m, multiplet), number of protons, coupling constants (J, Hz), and assignments.

### Dielectric Functions of Cage Molecule Thin Films Measured by Ellipsometry

To obtain the refractive index values required for effective medium theory analysis presented in the study, TP, COP-3P and COP-6VP molecules were spin-coated to thin films and ellipsometry was conducted. 1000 rpm spin speeds were used for all samples but, due to the slight differences in solution viscosity, the thicknesses varied slightly between 100 and 200 nm. The slight differences in thickness did not affect the experiment. Multiple measurements were conducted on the same material with different thicknesses, and the same refractive index values were obtained. The real part of the refractive index as a function of wavelength is illustrated in FIG. 6. All three types of molecules exhibited gradual dispersion resulting from the absorption band located in the UV region. In all cases, no measurable absorption was detected in the visible range.

### Example 1

### Synthesis of COP-1

To a solution of 1,3,5-trihexyl-2,4,6-tris(4-aminophenyl)benzene (428 mg, 0.71 mmol) and 10-bromo-1,8-diformylanthracene (335 mg, 1.07 mmol) in chloroform (237 mL) was added a solution of TFA (8 μL, 0.11 mmol) in CHCl₃ (100 μL) slowly dropwise. The solution was stirred at room temperature for 18 h, at which time ¹H NMR spectrum of the concentrated crude reaction mixture indicated both of the starting materials were consumed.

The solution was cooled to −45° C., and DIBAL-H (21.3 mL, 21.3 mmol, 1.0 M in CH₂Cl₂) was added. After stirring at −45° C. for 30 min, the reaction was quenched with MeOH (1 mL), and saturated NaHCO₃ (100 mL) was added. The mixture was stirred at room temperature for 30 min, and the organic layer was separated. The aqueous solution was extracted with CHCl₃ (3×75 mL). The combined organics were dried over Na₂SO₄, and concentrated. The residue was dissolved in THF (5 mL), and slowly added to MeOH (150 mL) dropwise. The pure product precipitated as a yellow solid, and was collected by filtration (478 mg, 66%). ¹H NMR (500 MHz, CDCl₃) δ 9.12 (s, 3H), 8.63-8.44 (m, 6H), 7.64-7.52 (m, 12H), 7.17 (d, J=13.6 Hz, 6H), 6.95-6.79 (m, 12H), 6.65 (dd, J=8.1, 2.1 Hz, 6H), 4.82 (s, 12H), 3.67 (s, 6H), 2.38-2.06 (m, 12H), 1.29-1.20 (m, 12H), 1.07-0.97 (m, 12H), 0.95-0.79 (m, 24H), 0.68 (t, J=7.3 Hz, 18H); ¹³C NMR (101 MHz, CDCl₃) δ 146.5, 139.7, 139.4, 136.0, 133.0, 132.1, 131.1, 130.9, 130.4, 128.5, 127.1, 124.2, 120.2, 114.5, 111.4, 47.4, 32.2, 31.4, 31.3, 29.9, 22.6, 14.4; MS (MALDI) calc'd for C₁₃₂H₁₄₁Br₃N₆ ([M+]) 2051.88, found 2050.61.

### Example 2

### Synthesis of COP-3P

In a Schlenk tube, COP-1 (85 mg, 0.04 mmol), pyridine-4-boronic acid (33 mg, 0.27 mmol), Pd(PPh₃)₄ (7 mg, 0.006 mmol), and Na₂CO₃ (65 mg, 0.62 mmol) were added. The reaction apparatus was then evacuated and refilled with nitrogen. Toluene (1 mL), THF (1 mL), and H₂O (0.5 mL) were added.

The mixture was degassed three times before heating at 105° C. for 2 days. Dichloromethane (50 mL) was added, and the mixture was washed with satd. NaHCO₃ (30 mL), and brine (30 mL). The organic solution was dried over Na₂SO₄, and concentrated to give the crude product. Purification by flash column chromatography using 10% methanol in toluene (v/v) yielded COP-3P as a light yellow solid (61 mg, 73%). ¹H NMR (500 MHz, CDCl₃) δ 9.22 (s, 3H), 8.94-8.82 (m, 6H), 7.59 (d, J=6.7 Hz, 6H), 7.55 (d, J=8.8 Hz, 6H), 7.45-7.34 (m, 12H), 7.22 (dd, J=11.1, 5.5 Hz, 6H), 6.97-6.86 (m, 12H), 6.69 (dd, J=8.1, 2.4 Hz, 6H), 4.88 (s, 12H), 3.84 (s, 6H), 2.36-2.23 (m, 12H), 1.36-1.27 (m, 12H), 1.11-1.01 (m, 12H), 0.99-0.83 (m, 24H), 0.69 (t, J=7.3 Hz, 18H); ¹³C NMR (75 MHz, CDCl₃) δ 150.3, 148.0, 146.6, 139.8, 139.5, 135.9, 135.2, 133.1, 132.1, 130.4, 129.9, 126.7, 126.0, 120.6, 114.5, 111.3, 47.4, 32.3, 31.3, 30.0, 22.7, 14.4; MS (MALDI) calc'd for C₁₄₇H₁₅₃N₉ ([M+]) 2045.85, found 2045.33.

### Example 3

### Synthesis of COP-6VP

COP-2 was prepared as reported in Jin et al., 2011, J. Am. Chem. Soc. 133:6650-6658. In a Schlenk tube, COP-2 (100 mg, 0.03 mmol), 4-vinylpyridine (38 mg, 0.36 mmol) Pd(OAc)₂ (1 mg, 0.004 mmol) and P(PhMe)₃ (1 mg, 0.003 mmol) were added. The reaction apparatus was then evacuated and refilled with nitrogen three times. DMF (1 mL), and Et₃N (0.5 mL) were added and the mixture was degassed three times before heating at 160° C. for 18 h. The mixture was cooled to room temperature, and dichloromethane (50 mL) was added. The mixture was washed with water (30 mL), and brine (30 mL). The organic solution was dried over Na₂SO₄, and concentrated to give the crude product. Purification by flash column chromatography (gradient elution, 2% MeOH/PhMe→10% MeOH/PhMe) yielded COP-6VP as a light yellow solid (65 mg, 63%). ¹H NMR (500 MHz, CDCl₃) δ 8.57-8.55 (m, 18H) 7.60-7.22 (m, 39H), 7.05-6.99 (m, 18H), 6.70-6.64 (m, 12H), 4.39 (s, 12H), 4.09 (br s, 6H), 3.98 (t, J=6.5 Hz, 6H), 2.05-2.01 (m, 12H), 1.82-1.71 (m, 6H), 1.64-1.56 (m, 6H), 1.40-1.18 (m, 72H), 1.00-0.96 (m, 12H), 0.88-0.81 (m, 36H), 0.78-0.66 (m, 27H); MS (MALDI) calc'd for C₂₄₆H₂₈₃N₁₂O₃ ([M+H]+) 3455.24, found 3455.78.

### Example 4

### Gold Nanoparticle Synthesis

Gold nanoparticles were prepared by the solution-based procedure described in Storhoff et al., 1998, J. Am. Chem. Soc. 120:1959-1964; and Cai et al., 2002, Analyst 127:803. Stock solution of HAuCl₄.3 H₂O in water (25 mM, 1.6 mL) was added to 38.4 mL water in 125 mL Erlenmeyer flask while being vigorously stirred. After 2 minutes of stirring, the solution was heated to boil. Trisodium citrate in water (34 mM, 4 mL) was added and refluxed for 10 minutes. After cooling down, the colloidal suspension of nanoparticles with an average diameter of 14 nm was stored in 50 mL polypropylene tube.

Substrate Preparation:

Substrates used for self-assembly were prepared in the following manner. First, a glass substrate was soaked into 1% Alconox/1% NaOH solution and heated to boil for 1 hour. After rinsing with water and 0.1M HCl, the substrate was soaked in boiling H₂O₂ for 1 hour. After extensive rinsing, the substrate was dried at 100° C. Once the cleaning procedure was complete, the substrate was soaked into 1% solution of 3-aminopropyltriethoxysilane (APTS) in toluene for 3 hours, rinsed with toluene and ethanol, and then put in a vacuum oven at 150° C. overnight.

Self-Assembly of Gold Nanoparticles:

Self-assembly of gold nanoparticles was conducted in a layer-by-layer fashion. Gold nanoparticle solution and cage molecule solution were applied to the self-assembly surface by drop-casting. For drop-casting, silicone glue was used to prepare a confinement cell of approximately 3 mm in diameter on a glass substrate treated with APTS as described above. The confinement cell kept the gold nanoparticle and cage molecule solutions inside and thus forced the self-assembly to occur within a well-defined area. For self-assembly, 10 mL of gold nanoparticle solution was dispensed and kept for 1 hour in a humidified petri dish. The slide was then rinsed with water extensively and dried.

Subsequently, a 3 mL of cage molecule solution in DMF was drop-casted and kept for 1 hour in a petri dish containing a DMF-soaked filter paper. The sample was then rinsed with DMF and then water extensively and dried. These two procedures of applying gold nanoparticles and cage molecules on the self-assembly surface were repeated to obtain the desired number of layers of gold nanoparticles.

### Example 5

### Template Synthesis of Gold Nanoparticles with an Organic Molecular Cage

The results described herein demonstrate a novel cage-templated strategy for the synthesis of narrowly dispersed Au NPs through the use of a well-defined, discrete organic molecular cage functionalized with pendent interior thioether groups. These results demonstrate that the successful controlled synthesis of Au NPs is attributed to the combination of a well-defined cage scaffold and the interaction between Au NPs and pendant thioether groups that serve as the nucleation and stabilization sites for Au NPs grown inside the cage. Because the size of the Au NPs match reasonably well with the volume of the cage cavity, such cage-template strategies may be useful in the rational design of size- and shape-tailored Au NPs. Exterior functionalized molecular cages may be useful to direct Au NP assembly.

### Synthesis of COP-11

Compared to conventional monodentate ligands, which form thick insulating layers on the Au NP surface, cage molecules with multiple internal binding sites can not only serve as a protecting shell with minimum coverage, but also control the growth of Au NPs with spatially confined cavity that is large enough to accommodate nanoparticles. Au NPs of a certain size is expected to grow inside the cage, leading to the formation of a core/shell structure: with Au NP as the core and cage molecule as the shell.

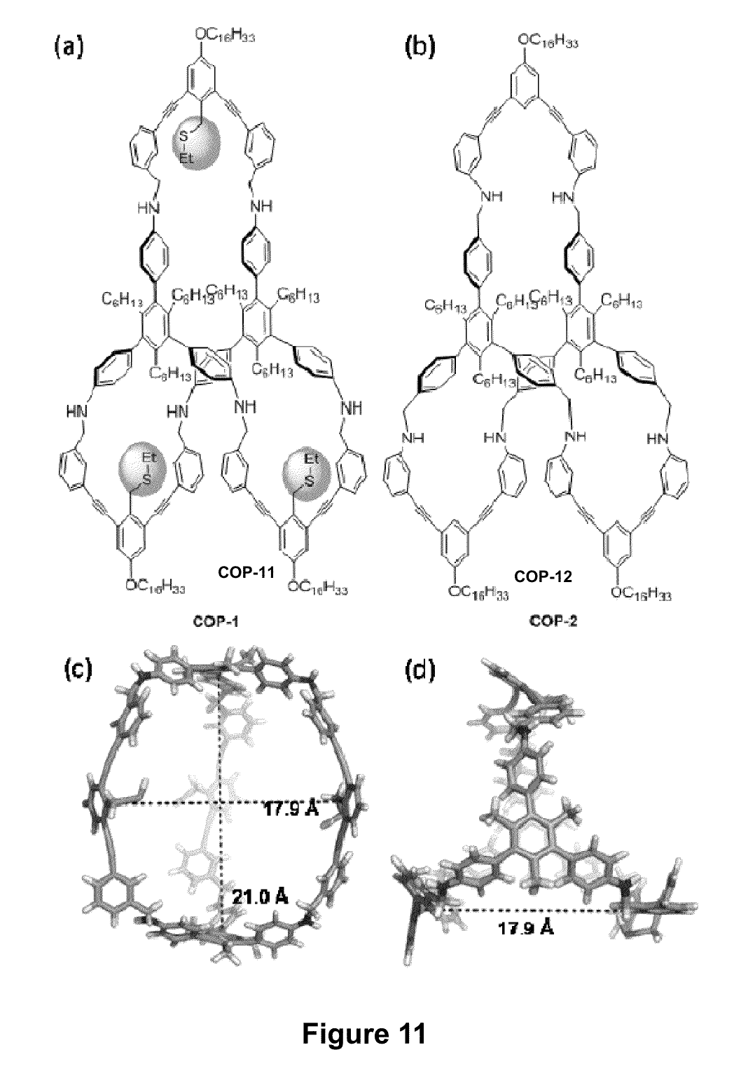

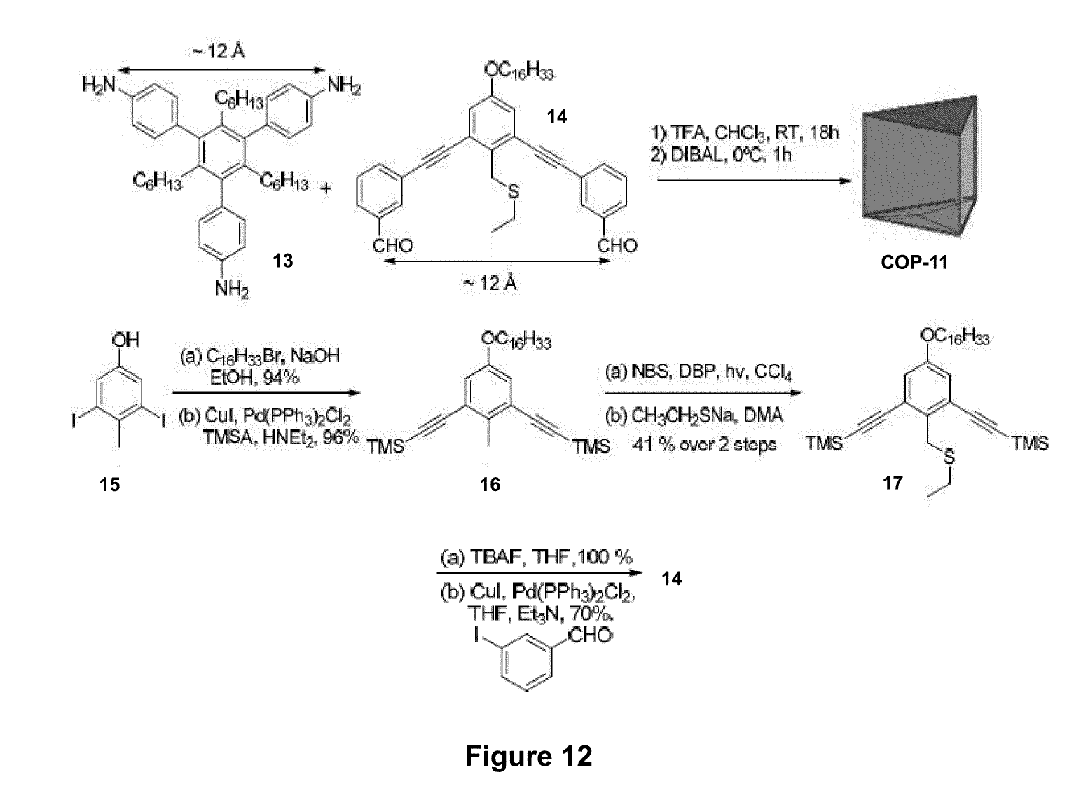

As a proof-of-concept, trigonal prismatic cage COP-11 with internal cavity size of 1.8-2.1 nm (FIG. 11) was designed, the interior of which is functionalized with three thioether groups. Thioether was used as the nucleation site for the Au deposition. Previously reported cage COP-12 was selected as a control compound (Zhang et al., 2011, J. Am. Chem. Soc. 133:6650-6658). COP-12 has the same cavity size as COP-11 but lacks the thioether anchoring groups. Triamine 13 was selected as the top and bottom panels and dialdehyde 14 was selected as the three lateral edges (FIG. 12). Triamine 13 was prepared using previously described methods (Jin et al., 2010, Angew. Chem. Int. Ed. 49:6348-6351). Dialdyhyde 14 was synthesized starting with 3,5-diiodo-p-cresol (15), which was prepared from p-cresol using previously described methods (Datta and Prosad, 1917, J. Am. Chem. Soc. 39:441-456). Following alkylation of compound 15, trimethylsilyl protected terminal acetylenes were introduced prior to radical bromination at the methyl position. The brominated intermediate was critical in that it allowed for the later introduction of pendant interior thioether groups. Desilylation of compound 17 followed by Sonogashira coupling with 3-iodo-benzaldyhyde yielded the lateral side piece 14. Imine condensation between the two building blocks 13 and 14 followed by subsequent reduction led to the formation of COP-11. COP-11 was characterized by ¹H NMR, gel permeation chromatography (GPC), and MALDI-MS.

### Synthesis of Cage-Encapsulated Gold Nanoparticles

Cage-encapsulated gold nanoparticles were prepared using a similar two-phase liquid-liquid approach to that previously reported by Brust et al, using tetraoctylammonium bromide (TOAB) as a phase-transfer reagent (Brust et al., 1994, Chem. Soc. Chem. Commun. 801-802). A solution of TOAB in CH₂Cl₂ was added to an aqueous solution of HAuCl₄ (10 equiv) and stirred until the aqueous layer was colorless, indicating that all AuCl₄⁻ was transferred to the organic phase. A solution of COP-11 (1 equiv) in CH₂Cl₂ was added to the above biphasic mixture. Upon mixing, no obvious color change was observed in the organic phase. The mixture was subsequently reduced with an aqueous solution of sodium borohydride (200 equiv, rt). The organic phase immediately changed color from orange-red to dark brown without any precipitates, indicating efficient Au³⁺ reduction and further stabilization of the resulting Au NPs by cage molecule COP-11. The organic layer containing AuNP@COP-11 complex was separated, and AuNP@COP-11 complex was precipitated out from ethanol and collected by centrifugation. The resulting AuNP@COP-11 complex was soluble in all organic solvents and stable in solution over periods of several weeks. The stable solution showed no evidence of agglomeration, and was without any noticeable color change. The Au NPs were characterized by UV-Vis and transmission electron microscopy (TEM).

The particle diameter and size distribution were analyzed based on the TEM images. All the samples were prepared using a solution of AuNP@COP-11 in CH₂Cl₂. The solution was drop cast on the carbon-coated 300 mesh copper grids (CF300-Cu), and allowed to air dry before being measured. The TEM image (FIG. 13A) of AuNP@COP-11 showed the formation of well-dispersed Au NPs, with an average diameter of 1.4 nm.

### Investigation of Cage Scaffold and Thioether Groups

In order to further investigate the role of cage scaffold and thioether groups in Au NP synthesis, two control experiments were conducted using COP-12 and thioether ligand 14. COP-12 is a structural analogue of COP-11 that lacks the three internal thioether groups of COP-11, while thioether ligand 14 lacks a cavity. First, HAuCl₄ was reduced in the presence of COP-12. Upon reduction with NaBH₄, the immediate and complete precipitation of aggregated NPs were observed. The TEM image of Au NPs obtained from the control experiment with COP-2 (FIG. 13B) showed only shapeless agglomerates, indicating that COP-12 lacks the requisite stability to serve as a template for the synthesis of Au NPs. Although not wishing to be bound by any particular theory, the reduced stability of COP-12 may be due to the lack of nucleation (i.e. Au binding) sites.

In another control experiment, when thioether ligand 14 was used as the ligand, similar rapid aggregation and precipitation of Au NPs upon reduction was observed. Although not wishing to be bound by any particular theory, this result suggests that Au NPs are poorly stabilized by monodentate open ligand 14. It was hypothesized that both the thioether groups and the closed cage structure itself are playing critical roles in controlled Au NP synthesis. The thioether groups may serve as the initial nucleation sites for Au NP growth and also stabilize the resulting nanoparticle. Once seeded, the Au nanocluster grows until it is confined sterically within the cage, and the three thioether groups and the six amino groups provide stabilization through Au surface binding. Although not wishing to be bound by any particular theory, the fact that the AuNP@COP-11 complexes themselves remain isolated from one another is likely due to the long alkyl chains present around the cage exterior.

### Computational Modeling Studies

To rationalize these experimental findings, computational simulations were used to model the interaction between COP-11 and Au NPs of different radii. The Au NPs were generated using cubic close-packed lattice of gold crystal structure with a closest Au—Au separation at 0.2884 nm. Five different Au NPs were used with radii of 11.54, 9.99, 8.65, 7.63, and 5.77 Å, respectively. For each nanoparticle, a cage was built around it and the three sulfur atoms of the cage were bonded to the gold atoms at the equator of the nanoparticle at angles of 120° from each other. The Amber 11.0 molecular dynamics program package was used to optimize the structures of the cage/nanoparticle complexes (Case et al., 2010, AMBER 11: University of California, San Francisco). The force field used for the cage was the general Amber force field (GAFF field)(Wang et al., 2004, J. Comput. Chem. 25:1157-1174) with the charge parameters computed by AM1-BCC method (Jakalian et al, 2000, J. Comput. Chem. 21:132-146).

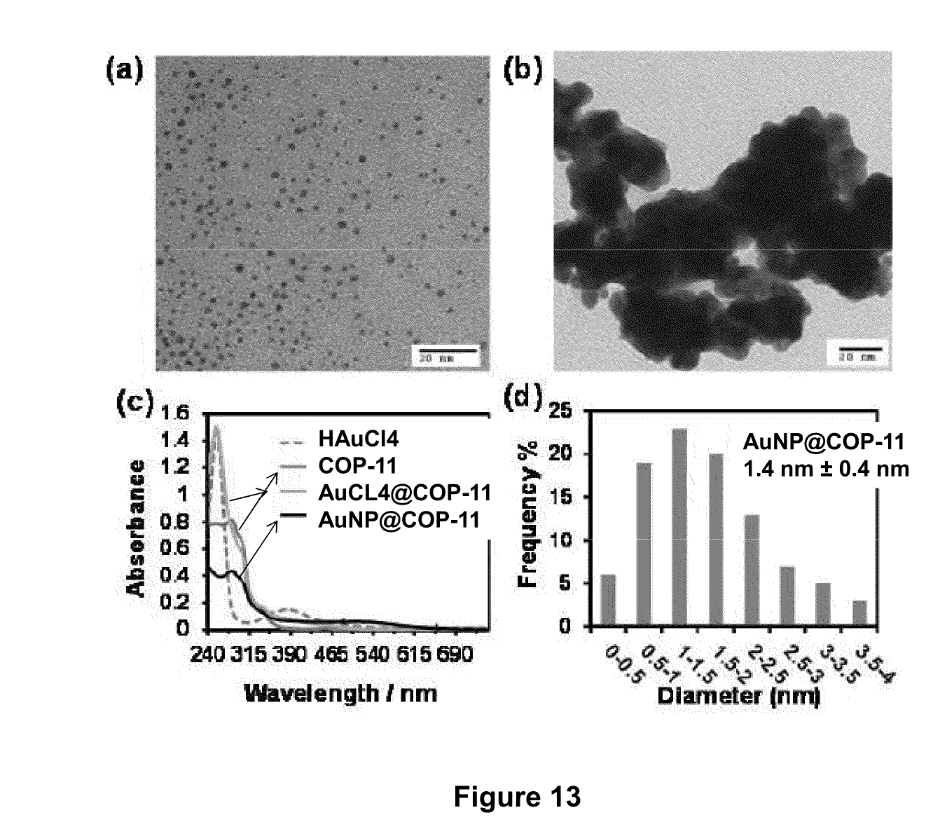

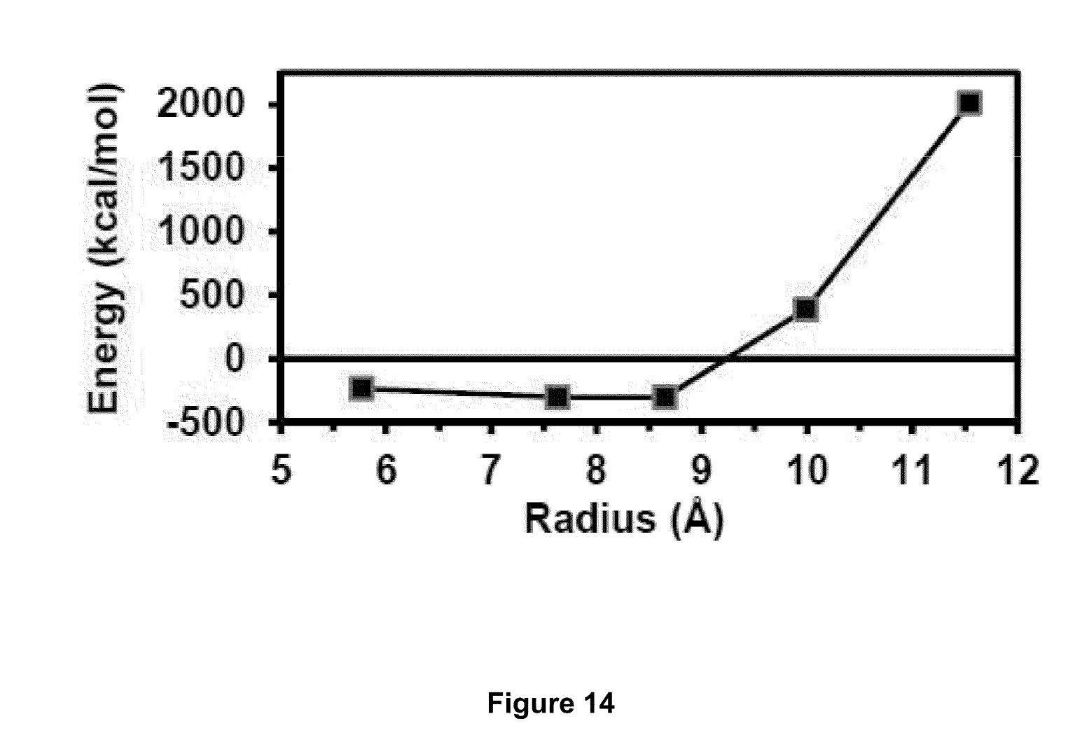

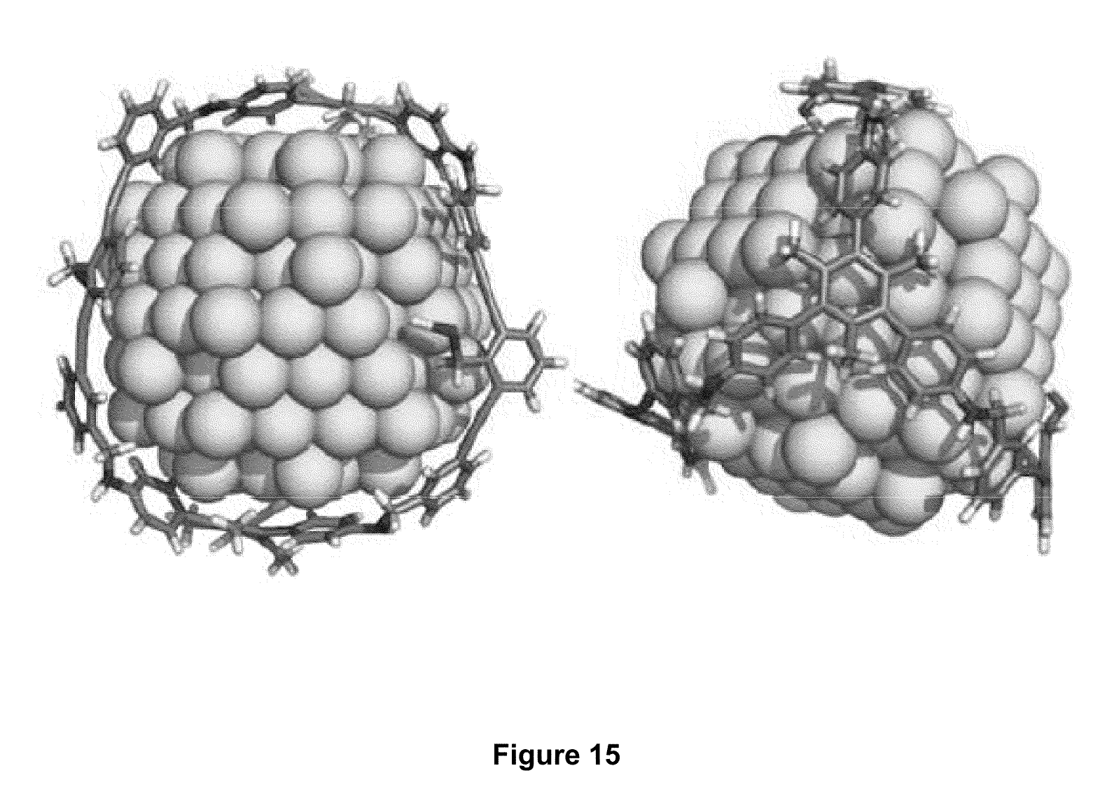

For each optimization run, the atoms on the gold nanoparticle were frozen, and the structure of the cage was optimized. The cage was first minimized for 5000 steps using the conjugate gradient method, and then it was further optimized by simulated annealing method for 150 picoseconds with a time-step of 1 femtosecond. During the simulated annealing, the system temperature was first raised up to 1000 K for 50 picoseconds and then gradually cooled to 0 K for another 100 picoseconds. Finally, the annealed structure was minimized again for another 5000 conjugate gradient steps. The total energy of cage-cage and cage-gold interactions was calculated based on the energy-minimized structure. FIG. 14 shows the cage energy as a function of the encapsulated nanoparticle radius. The energy first decreases when the radius increases due to the larger van der Waals attraction between the cage and the nanoparticle. At a radius between 7.63 and 8.65 Å, the energy reaches the minimum, and the structure of the AuNP@COP-11 complex at that energy minimum is presented in FIG. 15. The calculated structure in very close agreement with the 1.4±0.4 nm average diameter of the AuNP@COP-11 complex observed experimentally through the TEM characterization. When the nanoparticle radius is further increased, the cage has to stretch to accommodate the particles, causing the rapid increase in energy.

These results demonstrate that small Au NPs with narrow particle size distribution (1.4±0.4 nm) can be formed inside the cavity of an organic cage molecule. To the best of knowledge, this is the first example of in situ Au NP growth in a confined organic molecular environment. Moreover, the average particle size obtained from this cage-templated synthesis is consistent with the molecular dynamics simulation results. These results may be useful for guiding future rational design of particles of various sizes and different shapes. Furthermore, the abundance of available surface area from the resulting Au NP allows for the facile interactions of the Au NP with small molecules. Therefore, AuNP@COP-11 may be useful as a model complex for homogenous catalysis.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition comprising a shape-persistent three-dimensional cage molecule, wherein the cage molecule comprises two or more metal coordinating groups, whereby binding of nanoparticles to the two or more metal coordinating groups allows for the self-assembly of the nanoparticles into a nanocomposite material.

2. The composition of claim 1, wherein the nanoparticles comprise a metal nanoparticle, magnetic nanoparticle, fluorescent nanoparticle, semiconductor nanoparticle, quantum dot nanoparticle, dielectric nanoparticle, or any combinations thereof.

3. The composition of claim 1, wherein the nanocomposite material is deposited on at least a portion of the surface of a solid substrate.

4. The composition of claim 1, wherein the cage molecule is selected from the group consisting of formulas (I)-(III), a salt thereof and any combinations thereof: 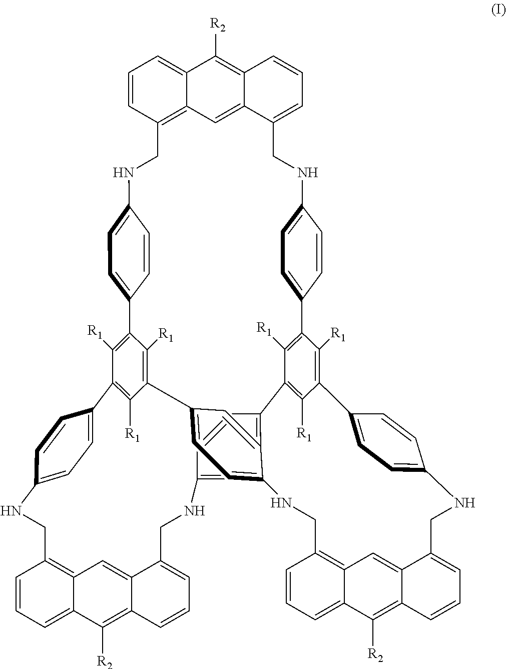 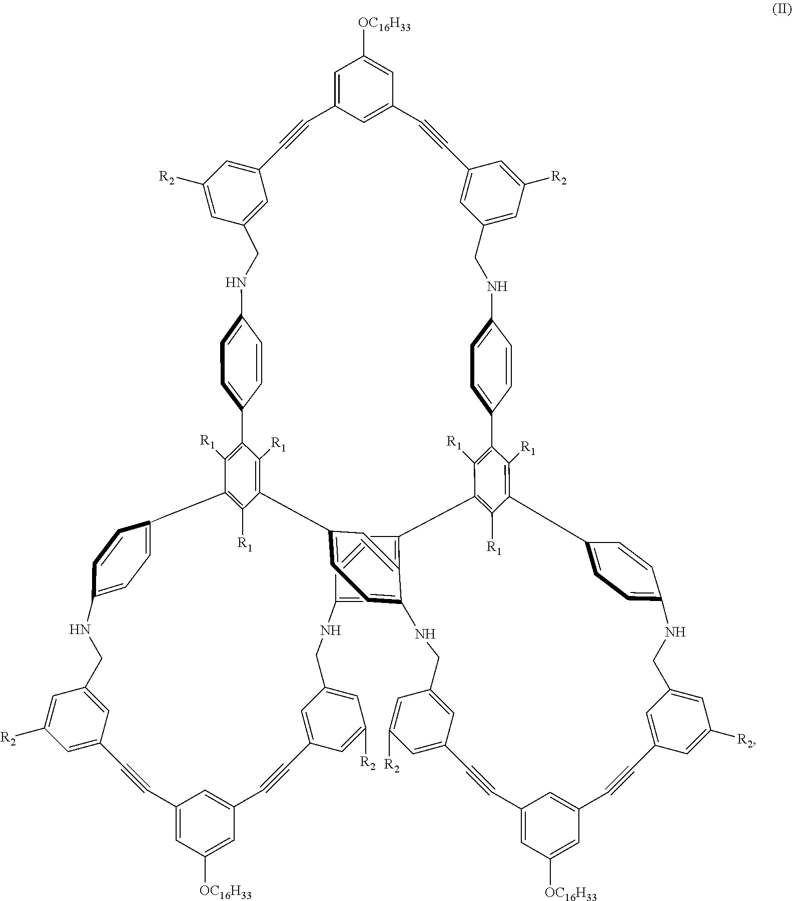
wherein in formulas (I)-(II):
each occurrence of R₁ is independently C₂-C₁₀ alkyl; and,
each occurrence of R₂ comprises a metal coordinating group and is independently selected from the group consisting of aryl, heteroaryl, arylalkyl, arylalkenyl, arylalkynyl, heteroarylalkyl, heteroarylalkenyl, heteroarylalkynyl, heterocyclyl, heterocyclylalkyl, heterocyclylalkenyl and heterocyclylalkynyl; and 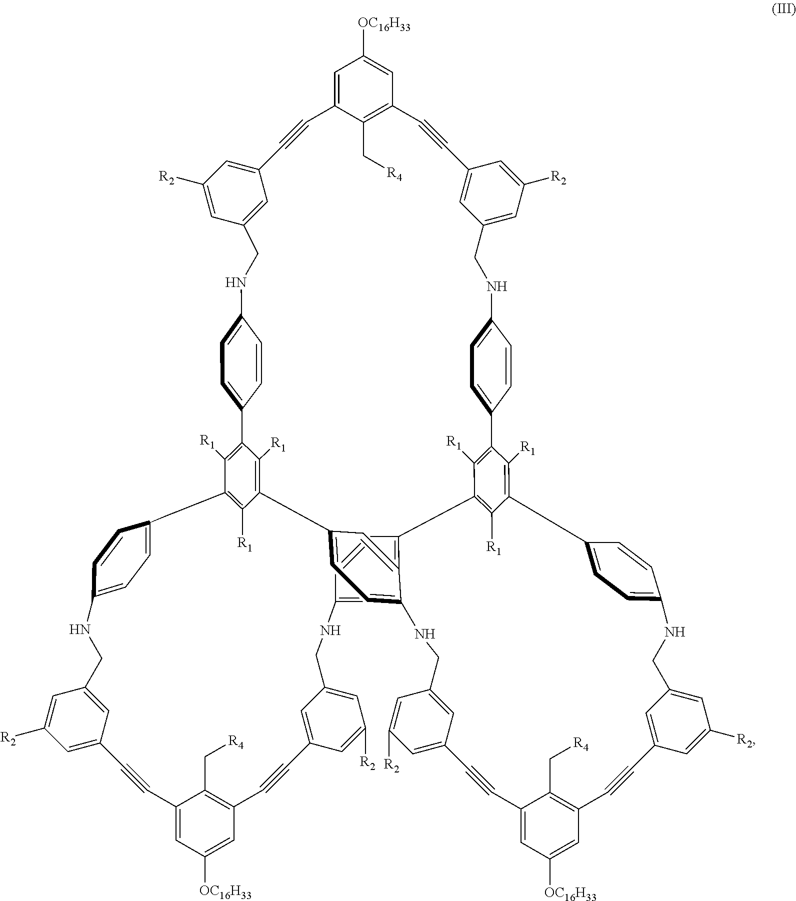
wherein in formula (III):
each occurrence of R₁ is independently C₂-C₁₀ alkyl;
each occurrence of R₂ is independently selected from the group consisting of hydrogen, aryl, heteroaryl, arylalkyl, arylalkenyl, arylalkynyl, heteroarylalkyl, heteroarylalkenyl, heteroarylalkynyl, heterocyclyl, heterocyclylalkyl, heterocyclylalkenyl and heterocyclylalkynyl; and,
each occurrence of R₄ is a metal coordinating group.

5. The composition of claim 4, wherein in formulas (I)-(III) each occurrence of R₁ is independently C₄-C₈ alkyl.

6. The composition of claim 4, wherein in formulas (I)-(II) the metal coordinating group in each occurrence of R₂ is independently selected from the group consisting of pyridyl, bipyridyl, terpyridyl, anilino, amino, carboxylate, phosphate, sulfate, amido, sulfonamido, hydroxy, sulfhydryl, and cyano.

7. The composition of claim 4, wherein in formula (III) each occurrence of R₂ is hydrogen.

8. The composition of claim 4, wherein in formula (III) each occurrence of R₄ is SR₃, wherein each occurrence of R₃ is independently H or C₁-C₁₀ alkyl.

9. The composition of claim 4, wherein the cage molecule comprises COP-3P, COP-6VP, COP-11, a salt thereof, or any combinations thereof.

10. The composition of claim 1, further comprising nanoparticles, wherein the nanoparticles are bound to the cage molecule through the two or more metal coordinating groups.

11. A method of preparing a self-assembling nanocomposite structure on at least a fraction of the surface of a solid substrate, the method comprising the steps of:
(a) providing a solid substrate;
(b) depositing a layer of nanoparticles on at least a fraction of the substrate surface;
(c) applying sequentially to the at least a fraction of the substrate surface a solution of a shape-persistent three-dimensional cage molecule and a solution of nanoparticles, wherein the cage molecule comprises two or more metal coordinating groups,
whereby binding of the nanoparticles to the two or more metal coordinating groups allows for the self-assembly of the nanoparticles into a nanocomposite material;
(d) optionally removing excess solution from the at least a fraction of the substrate surface; and,
(e) repeating steps (c) and (d) until the desired thickness of the self-assembling nanocomposite structure on the substrate surface is obtained.

12. The method of claim 11, wherein the nanoparticles comprise a metal nanoparticle, magnetic nanoparticle, fluorescent nanoparticle, semiconductor nanoparticle, quantum dot nanoparticle, dielectric nanoparticle, or any combinations thereof.

13. The method of claim 11, wherein step (b) comprises contacting the at least a fraction of the substrate surface with nanoparticles of opposite charge.

14. The method of claim 13, wherein the at least a fraction of the substrate surface is rendered positively charged by amine functionalization.

15. The method of claim 11, wherein the cage molecule is selected from the group consisting of formulas (I)-(III), any combinations thereof, and any salts thereof: 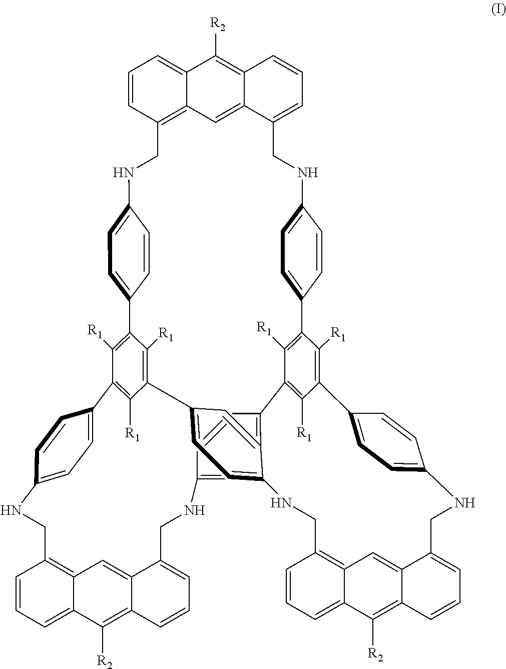 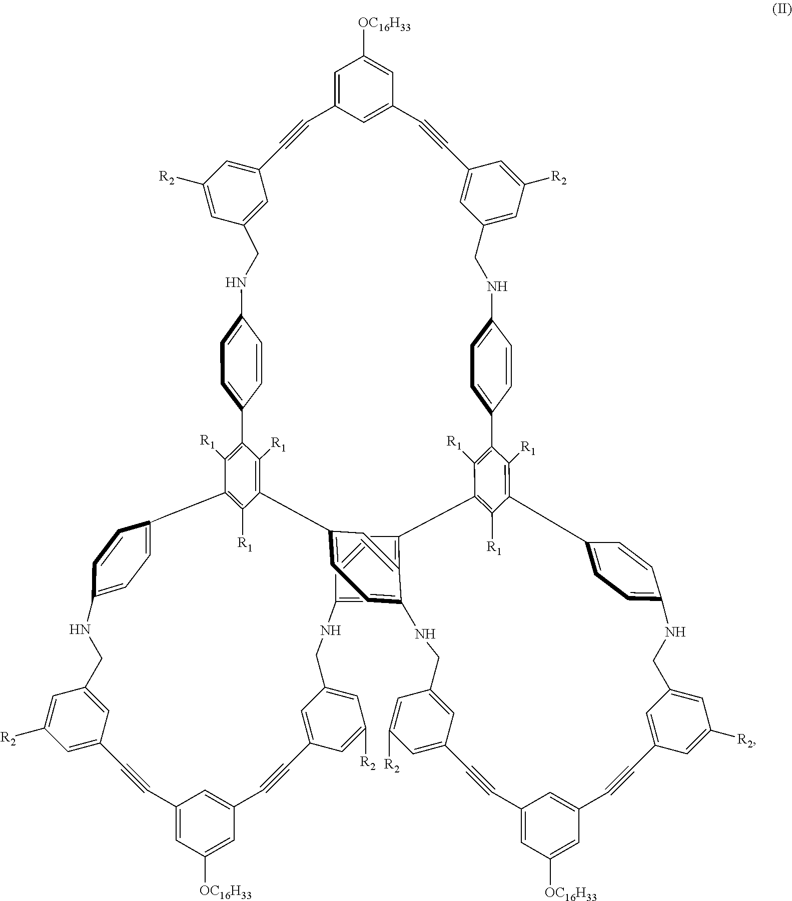
wherein in formulas (I)-(II):
each occurrence of R₁ is independently C₂-C₁₀ alkyl, and,
each occurrence of R₂ comprises a metal coordinating group and is independently selected from the group consisting of aryl, heteroaryl, arylalkyl, arylalkenyl, arylalkynyl, heteroarylalkyl, heteroarylalkenyl, heteroarylalkynyl, heterocyclyl, heterocyclylalkyl, heterocyclylalkenyl and heterocyclylalkynyl;
and 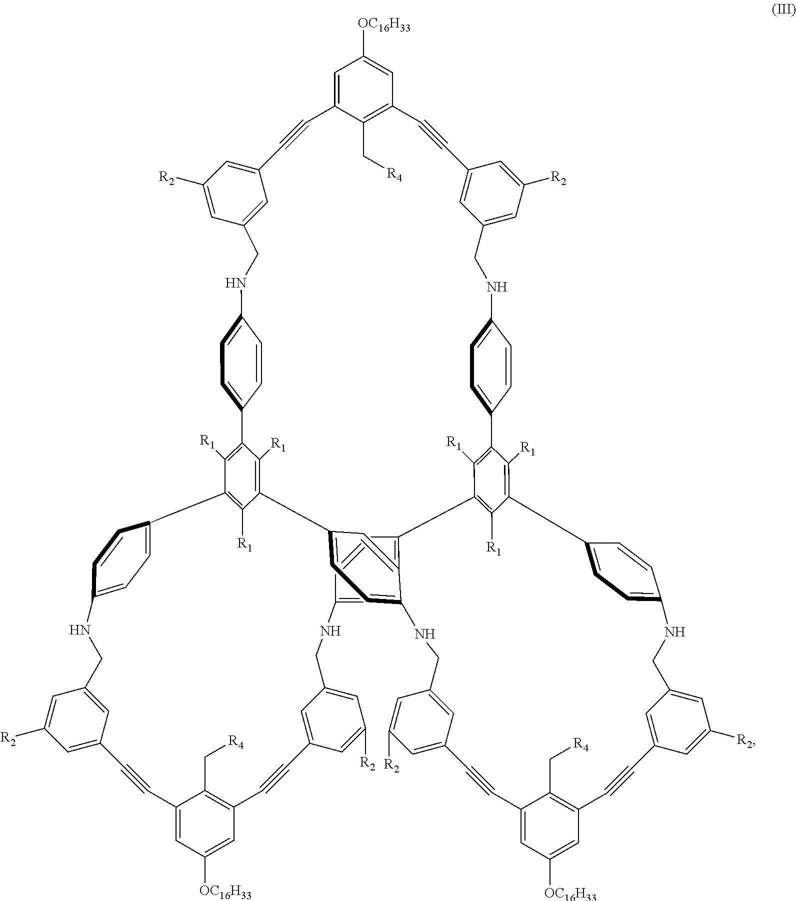
wherein in formula (III):
each occurrence of R₁ is independently C₂-C₁₀ alkyl,
each occurrence of R₂ is independently selected from the group consisting of hydrogen, aryl, heteroaryl, arylalkyl, arylalkenyl, arylalkynyl, heteroarylalkyl, heteroarylalkenyl, heteroarylalkynyl, heterocyclyl, heterocyclylalkyl, heterocyclylalkenyl and heterocyclylalkynyl, and,
each occurrence of R₄ is a metal coordinating group.

16. The method of claim 15, wherein in formulas (I)-(III) each occurrence of R₁ is independently C₄-C₈ alkyl.

17. The method of claim 15, wherein in formulas (I)-(II) the metal coordinating group in each occurrence of R₂ is independently selected from the group consisting of pyridyl, bipyridyl, terpyridyl, anilino, amino, carboxylate, phosphate, sulfate, amido, sulfonamido, hydroxy, sulfhydryl, and cyano.

18. The method of claim 15, wherein in formula (III) each occurrence of R₂ is hydrogen or each occurrence of R₄ is SR₃, wherein each occurrence of R₃ is independently H or C₁-C₁₀ alkyl.

19. The method of claim 15, wherein the cage molecule comprises COP-3P, COP-6VP, COP-11, a salt thereof or any combinations thereof.

20. The method of claim 11, wherein the metal nanoparticles comprises gold, silver, aluminum, copper, nickel, iron, chromium, titanium, platinum, cobalt, palladium, or any combinations thereof.
